(19) United States
(12) Patent Application Publication
(10) Pub. No.: US 2026/0325421 A1
(43) Pub. Date: Oct. 22, 2026

(54) LEAVE-ON PERSONAL CARE COMPOSITIONS HAVING AN EFFICIENT PERFUME ACCORD

(71) Applicant: THE PROCTER & GAMBLE COMPANY, Cincinnati, OH (US)

(72) Inventors: Jason John OLCHOVY, West Chester, OH (US); Virginia Tzung-Hwei HUTCHINS, Cincinnati, OH (US); Mara O'Brien GANNATTI, Cincinnati, OH (US)

(21) Appl. No.: 19/648,261

(22) Filed: Apr. 15, 2026

Related U.S. Application Data
(60) Provisional application No. 63/789,692, filed on Apr. 16, 2025.

(51) Int. Cl. A61K 8/37; A61K 8/26; A61K 8/28; A61K 8/33; A61K 8/34; A61K 8/35; A61K 8/49; A61Q 13/00; A61Q 15/00

(52) U.S. Cl. CPC A61K 8/37; A61K 8/26; A61K 8/28; A61K 8/33; A61K 8/342; A61K 8/35; A61K 8/4973; A61K 8/498; A61K 8/4986; A61Q 13/00; A61Q 15/00; A61K 2800/30; A61K 2800/591

(57) ABSTRACT

A leave-on personal care composition and methods of making a leave-on personal care composition that includes an active ingredient and a perfume accord. The perfume accord has an OIS of ≥6 and a tailored volatility distributions where about 10% to about 100%, by total weight of the perfume accord, of volatile materials each having a vapor pressure at 25 °C of less than 0.1 torr, from about 10% to about 90%, by total weight of the perfume accord, of volatile materials each having a vapor pressure at 25 °C of less than 0.01 torr, and from about 5% to about 70%, by total weight of the perfume accord, of volatile materials that each having a vapor pressure at 25 °C of less than 0.001 torr.

### FIELD

The present disclosure provides personal care compositions having efficient perfume bloom. The personal care compositions can include antiperspirant and deodorant compositions having perfume materials having high perceived odor intensity at low concentrations of perfume materials.

### BACKGROUND

Many consumers prefer personal care products, especially antiperspirants and deodorants, that feature a pleasant fragrance. The fragrance serves both functional and aesthetic purposes. Functionally, it helps to mask odors and enhances the effectiveness of the product by complementing its active ingredients, which work to neutralize odors. Aesthetically, the fragrance adds a pleasant scent that creates a feeling of freshness and well-being for the user. This combination of functionality and sensory appeal makes scented deodorants and antiperspirants particularly appealing to consumers.

However, the use of high levels of perfume compositions that result in a strong fragrance bloom in personal care products can present several challenges. First, elevated concentrations of fragrance, especially in leave-on personal care products, can increase the risk of skin irritation among users. Additionally, excessive fragrance levels can compromise product stability during storage, potentially leading to issues such as phase separation, discoloration, and the chemical degradation of both the fragrance and the active ingredients. Furthermore, the production of specialty perfumery chemicals is often associated with significant costs, making it economically advantageous to reduce fragrance concentrations in formulations.

Despite the challenges, reducing perfume composition levels without negatively impacting the intensity and quality of the fragrance is difficult, as significant reductions often lead to a marked decrease in fragrance intensity. Accordingly, there is a need for personal care compositions such as antiperspirants, deodorants, and other leave-on products comprising perfume materials that are more efficient, i.e., perfume materials that can provide a high perceived odor intensity when present in a product at low concentration.

### SUMMARY

A leave-on personal care composition comprising: (a) an active ingredient; (b) a perfume accord comprising perfume raw materials; wherein the perfume accord comprises: (i) an odor intensity score (OIS) of ≥6, as determined by Formula I:

$$1 / n {\sum }_{i = 1}^{n} {C}_{i} / {M}_{i} = OIS \qquad Formula ( I )$$

wherein n is the number of perfume raw materials present in the perfume accord, Cᵢ is the concentration of the i^{th} perfume raw material in the composition, and Mᵢ is the concentration of the i^{th} perfume raw material sufficient to provide an odor intensity rating in a leave-on composition chassis of 2 on a scale from zero to five; (ii) from about 10% to about 100%, by total weight of the perfume accord, of volatile materials each having a vapor pressure at 25 °C of less than 0.1 torr; (iii) from about 10% to about 90%, by total weight of the perfume accord, of volatile materials each having a vapor pressure at 25 °C of less than 0.01 torr (1.33 Pa); and (iv) from about 5% to about 70%, by total weight of the perfume accord, of volatile materials that each having a vapor pressure at 25 °C of less than 0.001 torr; wherein the leave-on composition chassis is the leave-on composition without the perfume accord.

A method of making a leave-on composition as described herein comprising (a) adding an active ingredient and optionally other ingredients; (b) adding the perfume accord; and (c) forming a leave-on composition.

### BRIEF DESCRIPTION OF THE DRAWINGS

While the specification concludes with claims particularly pointing out and distinctly claiming the composition, it is believed that this composition can be more readily understood from the following description taken in connection with the accompanying drawings, in which:

FIG. 1 shows two deodorant sticks with different levels of Perfume Accord A.

### DETAILED DESCRIPTION

Current perfume raw materials face several challenges including some consumers experiencing skin irritation from certain fragrances, especially those in leave-on compositions, like deodorants or antiperspirants, with high concentrations of perfume materials. Furthermore, it can be difficult to make a stable composition as perfume raw materials can lead to issues such as phase instability, crystallinity, and discoloration.

To address challenges related to skin irritation, physical stability, and cost, it can be desirable to use less perfume in personal care products, especially leave-on products. However, designing a perfume accord that maintains a consumer-preferred sensory experience and longevity at reduced concentrations is complex. Some olfactively desirable perfume materials when used at high levels can lead to skin irritation and are often minimized or avoided in formulations for leave-on products. However, incorporating these olfactively desirable perfume raw materials can be beneficial, it was found that more efficient perfume accords can contain a higher percentage of these perfume raw materials by weight as compared to current perfumes. The efficient perfume accords, described herein, can be physically stable and provide the sensory intensity and longevity that consumers desire.

In Table 1, the sensory intensity of Perfume Accord A (see Table 7) was compared to that of Perfume Accord B (see Table 8). Perfume Accord A was designed with an Odor Intensity Score (OIS) nearly twice as high as Perfume Accord B. Each perfume accord was compounded in Deodorant Chassis 1 (see Table 3) and rated on a scale of 0-100 (0 being very weak and 100 being very strong) at the initial application, 8 hours after application, and 24 hours after application. The sensory panel consisted of 15 to 20 naïve panelists, both male and female, aged 25 to 55. The ratings of each panel member were summed and divided by the number of members to provide a mean sensory intensity score, as shown in Table 1.

TABLE 1

| | | | Average | | |
|---|---|---|---|---|---|
| | | | Initial | | |
| | Perfume | | Application | Average 8- | Average 24- |
| Perfume | Level | OIS | Grade | Hour Grade | Hour Grade |
| Perfume | 0.50% | 11.0 | 70 | 55 | 40 |
| Accord A | | | | | |
| (see Table 7) | | | | | |
| Perfume | 4% | 5.2 | 70 | 50 | 40 |
| Accord B | | | | | |
| (see Table 8) | | | | | |

It was found because Perfume Accord A was designed with an OIS that was more than twice as large as Perfume Accord B, that even though the chassis had 8 times more Perfume Accord B, Perfume Accord A and B had similar scores for initial application, 8 hours, and 24 hours. Since Perfume Accord A is used at a much lower level, it is more efficient.

Furthermore, Perfume Accord A included 72% esters, by weight of the perfume accord, and Perfume Accord B included 11% esters, by weight of the perfume accord. Esters are common chemical moieties in perfume materials described as fruity, floral, and woody. However, they are known to hydrolyze and/or otherwise degrade in many types of formulations when used at high concentrations. As such, the overall percentage of esters in the product is generally limited, and they are used at a relatively low concentration within the perfume accord. Since Perfume Accord A is used at a much lower level in the chassis than Perfume Accord B, it allows for a higher percentage of esters to be included in Perfume Accord A as compared to Perfume Accord B.

It was found that high perfume levels can lead to product stability problems, such as crystallinity or discoloration. It is believed that perfume materials, such as esters, can hydrolyze or precipitate and cause crystallinity in the product. A deodorant product with crystals can have several issues that impact quality and user experience. Crystals may create a gritty texture, making the product uncomfortable to apply and difficult to apply evenly. They can also disrupt the uniform distribution of active ingredients, reducing effectiveness in odor control. Visually, crystals can appear unappealing, leading to perceptions of poor quality, as consumers expect a smooth product. Additionally, crystals can also clog the dispensing mechanism, especially for aerosol or roll-on antiperspirants, hindering usability.

The compositions described herein are physically stable. A composition is physically stable if by visual detection there the product appears uniform without crystals. As used herein, “visual detection” means that a human viewer can visually discern if the product had separated into 2 distinct layers or with crystals while being stored in a suitable package (e.g., a plastic or paperboard tube with a cap that is commonly used for stick deodorant products) with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm. The compositions described herein can also be color stable. A composition is color stable if Ab after storage for 5 days at 49 °C is less than 0.5, less than 0.25, or less than 0.10.

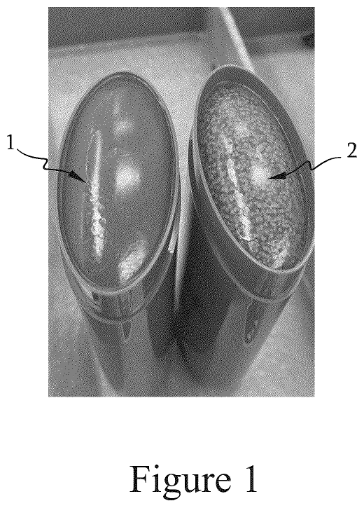

To determine whether the level of perfume raw materials impacted product stability, two examples were tested with the same chassis and perfume accord. After the products were made, they were stored for two weeks at ambient conditions in closed plastic deodorant tubes. Then, they were observed to see if crystals formed with visual detection. The results are shown in Table 2 and FIG. 1.

TABLE 2

| | | | Are crystals visible |
|---|---|---|---|
| | | Perfume Accord A | after 2 weeks at |
| | Chassis | (see Table 7) | ambient conditions? |
| Ex. 1 | Chassis 1 | 1.0% | No |
| | (see Table 3) | | |
| Ex. 2 | Chassis 1 | 4.0% | Yes |
| | (see Table 3) | | |

TABLE 3
Chassis 1

| | INCI |
|---|---|
| Old Spice ® High | Dipropylene Glycol, Water, Propylene Glycol, |
| Endurance Deodorant | Sodium Stearate, Poloxamine 1307, PPG-3 |
| | Myristyl Ether, Tetrasodium EDTA |

As shown in Table 2 and FIG. 1, Example 1 (stick 1 in FIG. 1) had 1% perfume accord and at this concentration the product passed the stability tests, showing no significant crystallinity or discoloration. However, Example 2 (stick 2 in FIG. 1) had 400 perfume accord, and it failed the stability test. It appears that the high level of esters from the 400 perfume accord hydrolyzed and formed crystals, as shown in stick 2. The color of stick 2 also changed. Therefore, it is desirable to formulate with a lower level of the perfume accord, to avoid these stability challenges.

Next, the same chassis and perfume accord was tested to determine whether the perfume accord could impact the color stability as determined by measuring and calculating the initial color and the end color and determining Δb, after storage for five days at 49 °C, representing accelerated stability conditions. Δb refers to color differences within the CIELAB color space where Ab represents the difference in the yellow/blue dimension calculated as the Euclidean distance between two colors in CIELAB space. The results are shown in Table 4, below.

TABLE 4

| | | Perfume Accord A | Δb after 5 days at |
|---|---|---|---|
| | Chassis | (see Table 7) | 49 °C. |
| Ex. 3 | Chassis 2 | 0.5% | 0.09 |
| | (see Table 5) | | |
| Ex. 4 | Chassis 2 | 2.0% | 0.83 |
| | (see Table 5) | | |

TABLE 5
Chassis 2

| | Active Ingredients | INCI |
|---|---|---|
| Gillette ® | Aluminum Zirconium | Water, Alcohol Denat., |
| Clear Gel | Octachlorohydrex Gly | Cyclopentasiloxane, Propylene |
| Antiperspirant | (anhydrous) 16.0% | Glycol. Dimethicone, Calcium |
| Deodorant | | Chloride, Peg/Ppg-18/18 |
| | | Dimethicone |

Example 3, which had 0.5% perfume accord, had significantly lower Ab, as compared to Example 4 and could be consumer preferred. A Ab of 0.83 indicates that the yellowing of the product would be visible to consumers, which is unattractive in a cosmetic product and consumers often believe that yellow products are no longer as effective.

Not only does the leave-on composition with the perfume accord need to be phase stable and color stable, and have a strong OIS score, but it also needs to providing lasting fragrance for extended durations, which is less important for rinse-off compositions. While not wishing to be bound by theory, the longevity of a perfume accord may be a function of various factors including vapor pressure (“VP”) of the volatile materials in the perfume accord. As used herein, the term “vapor pressure” means the partial pressure in air at a defined temperature for a given chemical species. It defines a chemical species' desire to be in the gas phase rather than the liquid or solid state. The higher the vapor pressure the greater the proportion of the material that will, at equilibrium, be found in a closed headspace. It is also related to the rate of evaporation of a flavor material which is defined in an open environment where material is leaving the system. The vapor pressure is determined according to the reference program Advanced Chemistry Development (ACD/Labs) Software Version 14.02, (© 1994-2015).

It was found that specific perfume accords with tailored volatility distributions can be effective, providing lasting fragrance for extended durations (e.g., ≥8 hours, ≥10 hours, ≥12 hours, ≥14 hours, ≥18 hours, ≥20 hours, and ≥24 hours) without degrading or losing effectiveness. Furthermore, these accords exhibit improved stability at lower concentrations and significantly reduce the risk of irritation and overall production costs while still delivering the desired sensory experience and longevity.

Table 6 shows the results of testing the % volatile material that have a vapor pressure of <0.1 torr, <0.01 torr, and <0.001 torr at 25 °C for Perfume Accord A (see Table 7), Perfume Accord B (see Table 8), and mint oil. The greater % of volatile material at lower vapor pressure indicates that the fragrance is long lasting, and it is important when designing fragrances with high OIS that they volatility distribution is still balanced to yield a long-lasting fragrance experience.

TABLE 6

| | | Perfume Accord A | Perfume Accord B | |
|---|---|---|---|---|
| | | (see Table 7) | (see Table 8) | Mint Oil |
| <0.1 | torr | 86.3% | 62.0% | 80.6% |
| <0.01 | torr | 71.9% | 46.3% | 0.5% |
| <0.001 | torr | 36.9% | 35.0% | 0.3% |

The results in Table 6 show that mint oil and Perfume Accord A both have a high number of volatile materials at <0.1 torr. However, the mint oil contains materials only with high volatility, limiting its long-lasting scent performance. Perfume Accord A and Perfume Accord B both have consumer preferred volatility distribution, indicating long-lasting fragrance.

The perfume accord may have from about 10% to about 100%, alternatively about 25% to about 98%, alternatively about 40% to about 95%, alternatively about 50% to about 95%, and alternatively about 60% to about 90%, by weight of the perfume accord, of volatile materials each having a VP at 25 °C of less than 0.1 torr (13.3 Pa). The perfume accord may have ≥65%, alternatively ≥70%, alternatively ≥75% alternatively ≥81%, alternatively ≥83%, and alternatively ≥85%, by weight of the perfume accord, of volatile materials that each having a VP at 25 °C of less than 0.1 torr (13.3 Pa).

The perfume accord may have from about 10% to about 90%, alternatively about 30% to about 85%, alternatively about 47% to about 80%, alternatively about 50% to about 78%, and alternatively about 60% to about 75%, by weight of the perfume accord, of volatile materials each having a VP at 25 °C of less than 0.01 torr (1.33 Pa). The perfume accord may have ≥1%, alternatively ≥10%, alternatively ≥30% alternatively ≥47%, alternatively ≥55%, alternatively ≥60%, alternatively ≥65%, and alternatively ≥70%, by weight of the perfume accord, of volatile materials that each having a VP at 25 °C of less than 0.01 torr (1.33 Pa).

The perfume accord may have from about 0.5% to about 70%, alternatively about 5% to about 60%, alternatively about 10% to about 50%, alternatively about 15% to about 45%, and alternatively about 30% to about 40%, by weight of the perfume accord, of volatile materials each having a VP at 25 °C of less than 0.01 torr (1.33 Pa). The perfume accord may comprise ≥0.5%, alternatively ≥1%, alternatively ≥5% alternatively ≥10%, alternatively ≥20%, alternatively ≥25%, alternatively ≥30%, and alternatively ≥35%, by weight of the accord, of volatile materials each having a VP at 25 °C of less than 0.001 torr (0.133 Pa).

### Perfume Accords

The compositions disclosed herein include a perfume accord and are suitable for leave-on application to the hair or skin. The perfume accords demonstrate a high perceived odor intensity (i.e., a strong scent) when present at low concentrations in the compositions and are capable of efficiently providing scents to the compositions.

The perfume accord can be a blend of perfume raw materials, as described herein, and comprises at least 10 perfume raw materials, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, and/or at least 30 perfume raw materials. The more perfume raw materials that make up a perfume accord, the more complex the perfume accord is.

The perfume accord is present in the compositions can be less than 3 wt. %, less than 2 wt. %, less than 1.75 wt. %, less than 1.5 wt. %, less than 1.25 wt. %, less than 1 wt. %, less than 0.9 wt. %, less than 0.8 wt. %, less than 0.7 wt. %, less than 0.6 wt. %, less than 0.5 wt. %, less than 0.4 wt. %, less than 0.3 wt. %, less than 0.2 wt. %, and/or less than 0.1 wt. % based on the total weight of the composition. In some embodiments the perfume accord can be present in the compositions at a concentration of from about 0.3 wt. % to about 1 wt. %, about 0.4 wt. % to about 1 wt. %, about 0.5 wt. % to about 0.9 wt. %, and/or about 0.6 wt. % to about 0.8 wt. % based on the total weight of the composition. The perfume accord can be present in the compositions at a concentration of from about 0.02 wt. % to about 0.3 wt. %, about 0.05 wt. % to about 0.3 wt. %, about 0.08 wt. % to about 0.25 wt. %, and/or about 0.1 wt. % to about 0.2 wt. % based on the total weight of the composition. The perfume accord can be present in the composition at a concentration from about 0.05% to about 5%, from about 0.21% to about 4%, from about 0.5% to about 3.5%, from about 0.75% to about 3%, from about 1% to about 2.5%, from about 1.1% to about 2%, from about 1.15% to about 1.5%.

The perfume accord can include from about 16.7% to about 100% esters, by weight of the perfume accord, alternatively from about 20% to about 95%, alternatively from about 25% to about 90%, alternatively from about 33% to about 85%, alternatively from about 50% to about 80%, alternatively from about 60% to about 75%. The perfume accord can include ≥12% esters, by weight of the perfume accord, alternatively ≥15%, alternatively ≥16.7%, alternatively ≥20%, alternatively ≥30%, alternatively ≥40%, alternatively ≥50%, alternatively ≥60%, and alternatively ≥65%.

The perfume accord disclosed herein may not be encapsulated. Alternatively, all or at least a portion of the perfume accord may be encapsulated. The compositions can include perfume materials in addition to the perfume accord, and these additional perfume materials can be encapsulated. A wide variety of capsules exist which will allow for delivery of perfume effect at various times. Examples of such capsules with different encapsulated materials are capsules provided by microencapsulation. One method comprises a capsule core which is coated completely with a material which may be polymeric. See, U.S. Pat. Nos. 4,145,184 and 4,234,627.

A wide variety of chemicals are known for fragrance (i.e., perfume) uses, including materials such as aldehydes, ketones and esters. More commonly, naturally occurring plant and animal oils and exudates comprising complex mixtures of various chemical components are known for use as fragrances. The fragrances herein can be relatively simple in their compositions, comprising a single chemical, or can comprise highly sophisticated complex mixtures of natural and synthetic chemical components, all chosen to provide any desired odor.

The perfume raw materials can have boiling points (BP) of about 500 °C or lower, more preferably about 400 °C or lower, even more preferably about 350 °C or lower. The BP of many perfume raw materials are given in Perfume and Flavor Chemicals (Aroma Chemicals), Steffen Arctander (1969). The C log P value of the perfume raw materials can be greater than about 0.1, greater than about 0.5, greater than about 1.0, and greater than about 1.2.

Suitable perfume raw materials can include any suitable perfume raw material, in particular high efficiency perfume materials that can include 8-methyl-1-oxaspiro[4.5]decan-2-one, (1-butoxy-1-oxopropan-2-yl) butanoate, (1E)-1-(2,6,6-trimethylcyclohex-2-en-1-yl)hepta-1,6-dien-3-one, (1-methyl-2-((1,2,2-trimethylbicyclo(3.1.0)hexan-3-yl)methyl)cyclopropyl)methanol, (1R,2R,4R)-1,2,3,3-tetramethylbicyclo(2.2.1)heptan-2-ol, (1R,2S,5R)-2-isopropyl-5-methylcyclohexan-1-ol, (1R,3R,6S,7S,8S)-2,2,6,8-tetramethyltricyclo[5.3.1.03,8]undecan-3-ol, (1R,4R,6R,10S)-4,12,12-trimethyl-9-methylidene-5-oxatricyclo[8.2.0.04,6]dodecane, (1R,4S,4aS,6R,8aS)-4,8a,9,9-tetramethyloctahydro-1,6-methanonaphthalen-1(2H)-ol, (1R,8R,10S,13R)-5,5,7,9,9,13-hexamethyl-4,6-dioxatetracyclo[6.5.1.01,10.03,7]tetradecane, (1R,9S,Z)-4,11,11-trimethyl-8-methylenebicyclo(7.2.0)undec-4-ene, (1S,2S,3S,5R)-2,6,6-trimethylspiro[bicyclo[3.1.1]heptane-3,4′-cyclohex-2-ene]-1′-one, (2-(cyclohexyloxy)ethyl)benzene, (2-(isopentyloxy)ethyl)benzene, (2,2-dimethoxyethyl)benzene, (2,4,6-trimethylcyclohex-3-en-1-yl)methanol, (2,5-dimethyl-1,3-dihydroinden-2-yl)methanol, (2E)-1,1-diethoxy-3,7-dimethylocta-2,6-diene, (2E)-3,7-dimethylocta-2,6-diene-1-thiol, (2E,6E)-3,7,11-trimethyldodeca-2,6,10-trien-1-ol, (2E,6Z)-nona-2,6-dien-1-ol, (2E,6Z)-nona-2,6-dienal, (2-methoxyethyl)benzene, (2S,4R)-2-methyl-4-propyl-1,3-oxathiane, (2S,5R)-2-isopropyl-5-methylcyclohexan-1-one, (2S,5S)-2-isopropyl-5-methylcyclohexan-1-one, (2Z,6E)-3,7-dimethylnona-2,6-dienenitrile, (3aR,5aR,9aR,9bR)-3a,6,6,9a-tetramethyldodecahydronaphtho(2,1-b)furan, (3aR,5aS,9aS,9bR)-3a,6,6,9a-tetramethyl-2,4,5,5a,7,8,9,9b-octahydro-1H-benzo[e][1]benzofuran, (3E,5E)-undeca-1,3,5-triene, (3E,5Z)-undeca-1,3,5-triene, (3E,6E)-nona-3,6-dien-1-ol, (3R,3aS,6R,7R,8aS)-3,6,8,8-tetramethyloctahydro-1H-3a,7-methanoazulen-6-ol, (4aR,5R,7aS,9R)-2,2,5,8,8,9a-hexamethyloctahydro-4H-4a,9-methanoazuleno(5,6-d)(1,3)dioxole, (4-methylphenyl) 2-methylpropanoate, (4-methylphenyl) 2-phenylacetate, (6E)-7,11-dimethyl-3-methylidenedodeca-1,6,10-triene, (E)-1,1-diethoxy-3,7-dimethylocta-2,6-diene, (E)-1,1-dimethoxy-3,7-dimethylocta-2,6-diene, (E)-1-methyl-4-(6-methylhept-5-en-2-ylidene)cyclohex-1-ene, (E)-2-(3,7-dimethylocta-2,6-dien-1-yl)cyclopentan-1-one, (E)-2,2-dimethyl-3-(3-methylpenta-2,4-dien-1-yl)oxirane, (E)-2,6,10-trimethylundeca-5,9-dienal, (E)-3,3-dimethyl-5-(2,2,3-trimethylcyclopent-3-en-1-yl)pent-4-en-2-ol, (E)-3,7,11-trimethyldodeca-1,6,10-trien-3-ol, (E)-3,7-dimethylocta-2,6-dien-1-yl (Z)-2-methylbut-2-enoate, (E)-3,7-dimethylocta-2,6-dien-1-yl 2-phenylacetate, (E)-3,7-dimethylocta-2,6-dien-1-yl acetate, (E)-3,7-dimethylocta-2,6-dien-1-yl butyrate, (E)-3,7-dimethylocta-2,6-dien-1-yl formate, (E)-3,7-dimethylocta-2,6-dienal, (E)-3-methyl-5-(2,2,3-trimethylcyclopent-3-en-1-yl)pent-4-en-2-ol, (E)-3-methylcyclopentadec-4-en-1-one, (E)-3-methylcyclotetradec-5-en-1-one, (E)-3-phenylprop-2-en-1-ol, (E)-4-((3aS,7aS)-octahydro-5H-4,7-methanoinden-5-ylidene)butanal, (E)-4-(2,6,6-trimethylcyclohex-2-en-1-yl)but-3-en-2-one, (E)-4,8-dimethyldeca-4,9-dienal, (E)-4-methyl-5-(4-methylphenyl)pent-4-enal, (E)-4-methyldec-3-en-5-one, (E)-5-(but-2-en-2-yl)-2,2-dimethyltetrahydrofuran, (E)-5-methylhept-2-en-4-one, (E)-6,10-dimethylundeca-5,9-dien-2-yl acetate, (E)-cyclohexadec-5-en-1-one, (E)-cyclopentadec-4-en-1-one, (E)-dec-2-enal, (E)-dec-4-enal, (E)-dodec-2-enal, (E)-hex-2-enal, (E)-hex-3-en-1-yl (E)-hex-3-enoate, (E)-oct-2-en-4-one, (E)-oxacycloheptadec-10-en-2-one, (E)-undec-9-enal, (R,E)-2-methyl-4-(2,2,3-trimethylcyclopent-3-en-1-yl)but-2-en-1-ol, (R,Z)-1-(2,6,6-trimethylcyclohex-2-en-1-yl)pent-1-en-3-one, (S)-2,3,3-trimethyl-2,3-dihydro-1H-inden-1-one, (S)-3-mercaptohexyl acetate, (Z)-1-((1R,2S)-2,6,6-trimethylcyclohex-3-en-1-yl)but-2-en-1-one, (Z)-1-(2,6,6-trimethylcyclohex-1-en-1-yl)but-2-en-1-one, (Z)-1-(2,6,6-trimethylcyclohex-2-en-1-yl)but-2-en-1-one, (Z)-1-(2,6,6-trimethylcyclohexa-1,3-dien-1-yl)but-2-en-1-one, (Z)-1-(benzyloxy)-2-methoxy-4-(prop-1-en-1-yl)benzene, (Z)-1,2-dimethoxy-4-(prop-1-en-1-yl)benzene, (Z)-1-methoxy-4-(prop-1-en-1-yl)benzene, (Z)-2-ethoxy-5-(prop-1-en-1-yl)phenol, (Z)-2-methoxy-4-(prop-1-en-1-yl)phenol, (Z)-2-methoxy-4-(prop-1-en-1-yl)phenyl acetate, (Z)-2-methylpent-2-enoic acid, (Z)-3,7-dimethylocta-1,3,6-triene, (Z)-3,7-dimethylocta-2,6-dien-1-ol, (Z)-3,7-dimethylocta-2,6-dien-1-yl acetate, (Z)-3-methyl-2-(pent-2-en-1-yl)cyclopent-2-en-1-one, (Z)-3-methyl-4-(2,6,6-trimethylcyclohex-2-en-1-yl)but-3-en-2-one, (Z)-4-(2,5,6,6-tetramethylcyclohex-2-en-1-yl)but-3-en-2-one, (Z)-5-methylheptan-3-one oxime, (Z)-cyclooct-4-en-1-yl methyl carbonate, (Z)-dodec-2-enal, (Z)-hex-3-en-1-ol, (Z)-hex-3-en-1-yl acetate, (Z)-hex-3-en-1-yl benzoate, (Z)-hex-3-en-1-yl butyrate, (Z)-hex-3-en-1-yl cyclopropanecarboxylate, (Z)-hex-3-en-1-yl formate, (Z)-hex-3-en-1-yl isobutyrate, (Z)-hex-3-en-1-yl methyl carbonate, (Z)-non-6-en-1-ol, (Z)-tridec-2-enenitrile, [(Z)-hex-3-enyl](E)-2-methylbut-2-enoate, [(Z)-hex-3-enyl]2-methylbutanoate, 1-((2E,5Z,9Z)-2,6,10-trimethylcyclododeca-2,5,9-trien-1-yl)ethan-1-one, 1-(2,2,6-trimethylcyclohexyl)hexan-3-ol, 1-(2,6,6-trimethylcyclohex-2-en-1-yl)pentan-3-one, 1-(3,3-dimethylcyclohexyl)ethan-1-one, 1-(3,3-dimethylcyclohexyl)ethyl formate, 1-(3-methylbenzofuran-2-yl)ethan-1-one, 1-(4-isopropylcyclohexyl)ethan-1-ol, 1-(4-methoxyphenyl)ethan-1-one, 1-(5,5-dimethylcyclohex-1-en-1-yl)pent-4-en-1-one, 1-(5,5-dimethylcyclohexen-1-yl)pent-4-en-1-one, 1-(cyclopropylmethyl)-4-methoxybenzene, 1-(naphthalen-2-yl)ethan-1-one, 1-(p-tolyl)ethan-1-one, 1-(spiro(4.5)dec-7-en-7-yl)pent-4-en-1-one, 1,1,2,3,3-pentamethyl-1,2,3,5,6,7-hexahydro-4H-inden-4-one, 1,1,5,5-tetramethylhexahydro-2H,5H-spiro((1,3)dioxolane-2,8-(2,4a)methanonaphthalene), 1,1,5,5-tetramethylhexahydro-2H-2,4a-methanonaphthalen-8(5H)-one, 1,3,3-trimethylbicyclo(2.2.1)heptan-2-yl acetate, 1,3,3-trimethylbicyclo[2.2.1]heptan-2-ol, 1,3-benzodioxole-5-carbaldehyde, 1,5-dimethylbicyclo(3.2.1)octan-8-one oxime, 1,7,7-trimethylbicyclo(2.2.1)heptan-2-ol, 1,7,7-trimethylbicyclo(2.2.1)heptan-2-one, 1,7-dioxacycloheptadecan-8-one, 1-butylsulfanylbutane, 1H-indole, 1-isopropyl-4-methylcyclohex-3-en-1-ol, 1-isopropyl-4-methylcyclohexa-1,4-diene, 1-methoxy-4-methylbenzene, 1-methoxy-4-propylbenzene, 1-methyl-4-(4-methylpent-3-en-1-yl)cyclohex-3-ene-1-carbaldehyde, 1-methyl-4-(4-methylpentyl)cyclohex-3-ene-1-carbaldehyde, 1-methylcyclohexa-1,3-diene, 1-phenylethyl acetate, 2-((3,7-dimethyloct-6-en-1-yl)oxy)acetaldehyde, 2-(1-ethoxyethoxy)ethylbenzene, 2-(2-hydroxypropan-2-yl)-5-methylcyclohexan-1-ol, 2-(2-hydroxypropoxy)propan-1-ol, 2-(2-mercaptopropan-2-yl)-5-methylcyclohexan-1-one, 2-(3,8-dimethyl-1,2,3,4,5,6,7,8-octahydroazulen-5-yl)propan-2-yl acetate, 2-(3-phenylpropyl)pyridine, 2-(4-methylcyclohex-3-en-1-yl)propan-2-ol, 2-(4-methylcyclohex-3-en-1-yl)propane-2-thiol, 2-(4-methylthiazol-5-yl)ethan-1-ol, 2-(8-isopropyl-6-methylbicyclo(2.2.2)oct-5-en-2-yl)-1,3-dioxolane, 2-(8-tricyclo[5.2.1.02,6]decanyl)acetaldehyde, 2-(p-tolyloxy)acetaldehyde, 2-(sec-butyl)cyclohexan-1-one, 2-(tert-butyl)-5-methyl-2-propyl-2,5-dihydrofuran, 2-(tert-butyl)cyclohexan-1-ol, 2,2,5-trimethyl-5-pentylcyclopentan-1-one, 2,2,6,6,7,8,8-heptamethyldecahydro-2H-indeno(4,5-b)furan, 2,2,6-trimethyl-6-vinyltetrahydro-2H-pyran, 2,2-dimethyl-3-methylenebicyclo(2.2.1)heptane, 2,2-dimethyl-4-phenylpentanenitrile, 2,3,5-trimethylpyrazine, 2,4a,5,8a-tetramethyl-1,2,3,4,4a,7,8,8a-octahydronaphthalen-1-yl formate, 2,4-dimethyl-2-(5,5,8,8-tetramethyl-5,6,7,8-tetrahydronaphthalen-2-yl)-1,3-dioxolane, 2,4-dimethyl-4-phenyltetrahydrofuran, 2,4-dimethylcyclohex-3-ene-1-carbaldehyde, 2,5,5-trimethyl-1,3,4,4a,6,7-hexahydronaphthalen-2-ol, 2,5,6-trimethylcyclohex-3-ene-1-carbaldehyde, 2,6,10-trimethylundec-9-enal, 2,6,6-trimethylbicyclo(3.1.1)hept-2-ene, 2,6-dimethylhept-5-enal, 2,6-di-tert-butyl-4-methylphenol, 2-benzyl-4,4,6-trimethyl-1,3-dioxane, 2-cyclododecylpropan-1-ol, 2-ethoxy-2,6,6-trimethyl-9-methylidenebicyclo[3.3.1]nonane, 2-ethoxy-4-methylphenol, 2-ethoxynaphthalene, 2-ethyl-3-hydroxy-4H-pyran-4-one, 2-ethyl-4-hydroxy-5-methylfuran-3(2H)-one, 2-ethyl-N-methyl-N-(m-tolyl)butanamide, 2-heptylcyclopentan-1-one, 2-hexylcyclopent-2-en-1-one, 2-hydroxy-3-methylcyclopent-2-en-1-one, 2-isobutyl-3-methoxypyrazine, 2-isobutylthiazole, 2-isopropyl-4-methylthiazole, 2-isopropyl-5-methylphenol, 2-methoxy-4-(methoxymethyl)phenol, 2-methoxy-4-propylphenol, 2-methyl-1-phenylpropan-2-ol, 2-methyl-1-phenylpropan-2-yl butyrate, 2-methyl-3-((1,7,7-trimethylbicyclo(2.2.1)heptan-2-yl)oxy)propan-1-ol, 2-methyl-4-oxo-4H-pyran-3-yl isobutyrate, 2-methyl-4-phenylbutan-2-ol, 2-methyl-5-(prop-1-en-2-yl)cyclohex-2-en-1-one, 2-methyl-5-phenylpentan-1-ol, 2-methyl-6-methyleneoct-7-en-2-yl acetate, 2-methyldecanal, 2-methyldecanenitrile, 2-pentylcyclopentan-1-ol, 2-pentylcyclopentan-1-one, 2-phenylacetaldehyde, 2-phenylethanethiol, 2-phenylethyl (E)-2-methylbut-2-enoate, 2-phenylpropanal, 3-(2-ethylphenyl)-2,2-dimethylpropanal, 3-(3,3-dimethyl-2,3-dihydro-1H-inden-5-yl)propanal, 3-(3,3-dimethyl-2,3-dihydro-1H-inden-5-yl)propanal mixture with 3-(1,1-dimethyl-2,3-dihydro-1H-inden-5-yl)propanal, 3-(3-isopropylphenyl)butanal, 3-(4-(tert-butyl)phenyl)propanal, 3-(4-ethylphenyl)-2,2-dimethylpropanenitrile, 3-(4-methoxyphenyl)-2-methylpropanal, 3-(4-methylcyclohex-3-en-1-yl)butan-1-ol, 3-(4-methylcyclohex-3-en-1-yl)butanal, 3-(6,6-dimethylbicyclo(3.1.1)hept-2-en-2-yl)-2,2-dimethylpropanal, 3-(6,6-dimethylbicyclo(3.1.1)hept-2-en-2-yl)propanal, 3,4-diethyl-2,5-dimethylcyclohex-2-en-1-one, 3,4-dimethoxybenzaldehyde, 3,5,10,10-tetramethylspiro[5.5]undec-3-en-1l-one, 3,7-dimethyl-2-methylideneoct-6-enal, 3,7-dimethyloct-6-en-1-ol, 3,7-dimethyloct-6-en-1-yl acetate, 3,7-dimethyloct-6-en-1-yl formate, 3,7-dimethyloct-6-enal, 3,7-dimethyloct-6-enenitrile, 3,7-dimethylocta-1,6-dien-3-yl benzoate, 3,7-dimethylocta-1,6-dien-3-yl isobutyrate, 3,7-dimethylocta-1,6-dien-3-yl propionate, 3,7-dimethyloctan-1-ol, 3,7-dimethyloctanal, 3,7-dimethyloctane-1,7-diol, 3a,6,6,9a-tetramethyl-2,4,5,5a,7,8,9,9b-octahydro-1H-benzo[e][1]benzofuran, 3-ethoxy-1,1,5-trimethylcyclohexane, 3-ethoxy-4-hydroxybenzaldehyde, 3-hydroxy-2-methyl-4H-pyran-4-one, 3-hydroxybutan-2-one, 3-methyl-4-phenylbutan-2-ol, 3-methyl-5-phenylpentanal, 3-methylbut-2-en-1-yl acetate, 3-methylbutan-1-ol, 3-methylbutyl acetate, 3-methylcyclopentadecan-1-one, 3-methylnonane-2,4-dione, 3-pentyltetrahydro-2H-pyran-4-yl acetate, 3-phenylpropan-1-ol, 4-(4-hydroxyphenyl)butan-2-one, 4-(4-Methyl-3-pentenyl)-3-cyclohexene-1-carboxaldehyde, 4-(tert-butyl)cyclohexan-1-ol, 4-(tert-pentyl)cyclohexan-1-one, 4,4a,5,9b-tetrahydroindeno(1,2-d)(1,3)dioxine, 4,6-dimethylcyclohex-3-ene-1-carbaldehyde, 4,6-dimethylpyran-2-one, 4,7,7-trimethyl-6-thiabicyclo[3.2.1]octane, 4,8-dimethyl-2-(propan-2-ylidene)-1,2,3,3a,4,5,6,8a-octahydroazulen-6-yl acetate, 4,9,12,12-tetramethyl-5-oxatricyclo(8.2.0.04,6)dodecane, 4-allyl-2-methoxyphenol, 4-formyl-2-methoxyphenyl isobutyrate, 4-hydroxy-2,3-dimethyl-2H-furan-5-one, 4-hydroxy-2,5-dimethylfuran-3-one, 4-hydroxy-3-methoxybenzaldehyde, 4-isopropylbenzaldehyde, 4-isopropylbenzonitrile, 4-mercapto-4-methylpentan-2-one, 4-methoxy-2-methylbutane-2-thiol, 4-methoxybenzaldehyde, 4-methoxybenzyl acetate, 4-methyl-2-(2-methylprop-1-en-1-yl)tetrahydro-2H-pyran, 4-methyl-2-(2-methylpropyl)oxane, 4-methyl-2-phenyl-3,6-dihydro-2H-pyran, 4-methyl-2-phenyltetrahydro-2H-pyran, 4-methyl-4-phenylpentan-2-yl acetate, 4-methylpent-4-en-2-yl isobutyrate, 4-prop-1-en-2-ylcyclohexene-1-carbaldehyde, 4-vinylcyclohex-1-ene-1-carbaldehyde, 5-(diethoxymethyl)benzo(d)(1,3)dioxole, 5-(sec-butyl)-2-(2,4-dimethylcyclohex-3-en-1-yl)-5-methyl-1,3-dioxane, 5-butyl-4-methyloxolan-2-one, 5-butyldihydro-2(3H)-furanone, 5-ethyldihydrofuran-2(3H)-one, 5-hexyl-5-methyldihydrofuran-2(3H)-one, 5-mercapto-5-methylhexan-3-one, 5-methyl-2-(2-sulfanylpropan-2-yl)cyclohexan-1-one, 5-methyloxolan-2-one, 5-pentyloxolan-2-one, 5-propyldihydrofuran-2(3H)-one, 6-(sec-butyl)quinoline, 6,6-dimethoxy-2,5,5-trimethylhex-2-ene, 6,6-dimethyl-2-methylenebicyclo(3.1.1)heptane, 6,8-dimethylnon-7-enal, 6-butyl-2,4-dimethyl-3,6-dihydro-2H-pyran, 6-heptyltetrahydro-2H-pyran-2-one, 6-isopropylquinoline, 6-methoxy-2,6-dimethylheptanal, 6-methyl-2-(oxiran-2-yl)hept-5-en-2-ol, 6-methylhept-5-en-2-one, 6-pentyltetrahydro-2H-pyran-2-one, 6-propyloxan-2-one, 7-methyl-2H-benzo(b)(1,4)dioxepin-3(4H)-one, 7-methyl-3-methyleneocta-1,6-diene, 8,8-di(1H-indol-1-yl)-2,6-dimethyloctan-2-ol, 8,8-dimethyl-1,2,3,4,5,6,7,8-octahydronaphthalene-2-carbaldehyde, 8-isopropyl-6-methylbicyclo(2.2.2)oct-5-ene-2-carbaldehyde, acetophenone, allyl 2-(cyclohexyloxy)acetate, allyl 2-(isopentyloxy)acetate, allyl 2-phenoxyacetate, allyl 3-cyclohexylpropanoate, allyl heptanoate, allyl hexanoate, benzaldehyde, benzo(d)(1,3)dioxole-5-carbaldehyde, benzophenone, benzyl butyrate, benzyl isobutyrate, cinnamaldehyde, cinnamonitrile, cinnamyl acetate, dec-9-en-1-ol, decanal, decanenitrile, dodecanal, dodecanenitrile, ethyl (1R,6S)-2,2,6-trimethylcyclohexane-1-carboxylate, ethyl (1S,2R,3R,4S)-3-isopropylbicyclo(2.2.1)hept-5-ene-2-carboxylate, ethyl (2Z,4E)-deca-2,4-dienoate, ethyl (3aR,4S,7R,7aR)-octahydro-3aH-4,7-methanoindene-3a-carboxylate, ethyl (3S)-3-hydroxybutanoate, ethyl 2,6,6-trimethylcyclohexa-1,3-diene-1-carboxylate, ethyl 2-ethyl-6,6-dimethylcyclohex-2-ene-1-carboxylate, ethyl 2-hydroxybenzoate, ethyl 2-methylbutanoate, ethyl 2-methylpentanoate, ethyl 3-methyl-3-phenyloxirane-2-carboxylate, ethyl 3-methylbutanoate, ethyl 3-oxobutanoate, ethyl 3-phenyloxirane-2-carboxylate, ethyl acetate, ethyl butyrate, ethyl heptanoate, ethyl hexanoate, ethyl octanoate, ethyl propionate, heptan-2-one, hexyl 2-methylbutanoate, hexyl isobutyrate, isobutyl acetate, isopentyl butyrate, isopentyl propionate, isopropyl 2-methylbutanoate, methyl (E)-2-(((3,5-dimethylcyclohex-3-en-1-yl)methylene)amino)benzoate, methyl (E)-2-((7-hydroxy-3,7-dimethyloctylidene)amino)benzoate, methyl (E)-non-2-enoate, methyl (Z)-2-((3-(4-(tert-butyl)phenyl)-2-methylpropylidene)amino)benzoate, methyl 2-(methylamino)benzoate, methyl 2,2-dimethyl-6-methylidenecyclohexane-1-carboxylate, methyl 2,4-dihydroxy-3,6-dimethylbenzoate, methyl 2-[3-oxo-2-[(E)-pent-2-enyl]cyclopentyl]acetate, methyl 2-aminobenzoate, methyl 2-cyclopentylideneacetate, methyl 2-hydroxybenzoate, methyl 2-phenylacetate, methyl benzoate, methyl cinnamate, methyl non-2-ynoate, methyl oct-2-ynoate, methylsulfanylmethane, N,2-dimethyl-N-phenylbutanamide, nona-2,6-dienenitrile, nonan-1-ol, nonanal, octahydro-1H-4,7-methanoindene-1-carbaldehyde, octan-1-ol, octan-2-one, octan-3-one, octanal, oxydibenzene, p-cymene, pentane-2,3-dione, pentyl acetate, phenethyl acetate, phenethyl formate, phenethyl isobutyrate, propane-1,2-diol, trideca-3,12-dienenitrile, triethyl 2-hydroxypropane-1,2,3-tricarboxylate, undec-10-enal, undecan-2-one, undecanal, or mixtures thereof.

### Odor Intensity Score

The perfume accord can have a high odor intensity score (OIS). The odor intensity score (OIS) of a composition is determined by Formula I:

$$1 / n {{\sum }^{n}}_{i = 1} {C}_{i} / {M}_{i} = OIS \qquad Formula ( I )$$

In Formula I, n is the number of perfume raw materials present in the perfume accord, Cᵢ is the concentration of the i^{th} perfume raw material in the composition, and Mᵢ is the concentration of the i^{th} perfume raw material sufficient to provide an odor intensity rating (OIR) in a composition chassis of 2 on a scale from zero to five. The composition chassis has the same formulation as the composition with the exception that the composition chassis does not include the perfume accord.

The odor intensity rating (OIR) is determined by a sensory panel rating the sensory intensity of a perfume raw material in a composition chassis (i.e., the composition without the perfume accord) on a scale from zero to five. The sensory panel consists of about 15 to about 20 naïve panelists, both male and female, aged 25 to 55. For each perfume raw material, the sensory panel rates at least two samples, one of which includes the perfume raw material at a concentration of about 10-times the odor detection threshold of the perfume raw material, and the other of which includes the perfume raw material at a concentration of about 250-times the odor detection threshold of the perfume raw material. The odor detection threshold of a perfume raw material is the concentration of the perfume raw material at which 50% of the population detects an odor above background. By way of example, if the odor detection threshold of a perfume raw material is 20 ppm, one sample includes the perfume raw material in the composition chassis at about 200 ppm and another sample includes the perfume raw material in the composition chassis at about 5000 ppm. An individual rating by a panel member of zero means that the individual could not detect an odor and an individual rating by a panel member of five means that the individual could detect an extremely strong odor. The ratings of each panel member at each concentration of perfume raw material are summed and divided by the number of members to provide a mean rating that defines the OIR at each concentration tested.

M is determined for each perfume raw material by using the OIR values (obtained as described above) to calculate the concentration of the perfume raw material in the composition chassis sufficient to provide an OIR of 2. The methods for determining M using a sensory panel include extrapolating or interpolating the OIR values to obtain a value for M. The methods for determining M using a sensory panel also include preparing samples using perfume raw materials at concentrations other than (or in addition to) about 10-times the odor detection threshold of the perfume raw material and about 250-times the odor detection threshold of the perfume raw material.

Without intending to be bound by theory, perfume raw materials having a low value for M typically demonstrate a high perceived odor intensity (i.e., a strong scent) when present in a composition at low concentration. Put another way, a first perfume raw material having a lower relative value for M than a second perfume raw material typically demonstrates a higher perceived odor intensity (i.e., a stronger scent) relative to the perceived odor intensity of the second perfume raw material when the first and second perfume raw materials are present in a composition at the same concentration. Thus, perfume accords having a high number of perfume raw materials with low M values advantageously provide a high perceived odor intensity (i.e., a strong scent) when present in a composition at low concentration. In some embodiments, about 20% to about 100% of the perfume raw materials present in a perfume accord have a value for M of about 1 ppm to about 1000 ppm, for example, about 30% to about 100%, about 40% to about 100%, about 50% to about 100%, about 60% to about 100%, and/or about 70% to about 100% of the perfume raw materials present in the perfume accord. In some embodiments, about 20% to about 100% of the perfume raw materials present in a perfume accord have a value for M of about 1 ppm to about 800 ppm, for example, about 30% to about 100%, about 40% to about 100%, about 50% to about 100%, about 60% to about 100%, and/or about 70% to about 100% of the perfume raw materials present in the perfume accord. In some embodiments, about 20% to about 100% of the perfume raw materials present in a perfume accord have a value for M of about 1 ppm to about 600 ppm, for example, about 30% to about 100%, about 40% to about 100%, about 50% to about 100%, about 60% to about 100%, and/or about 70% to about 100% of the perfume raw materials present in the perfume accord.

The compositions disclosed herein have a high percentage of perfume raw materials that contribute to the overall perception of the fragrance at a noticeable level. Without intending to be bound by theory, perfume raw materials contribute to the overall perception of the fragrance at a noticeable level when the ratio of the concentration of the perfume raw material in a composition to the M value of the perfume raw material in the corresponding composition chassis (i.e., C/M) is high. In some embodiments, Cᵢ/Mᵢ is at least 0.05 for about 75% to about 100% of the perfume raw materials present in the perfume accord, for example, about 80% to about 100%, and/or about 90% to about 100% of the perfume raw materials present in the perfume accord. In some embodiments, Cᵢ/Mᵢ is at least 0.1 for about 70% to about 100% of the perfume raw materials present in the perfume accord, for example, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.2 for about 45% to about 100% of the perfume raw materials present in the perfume accord, for example, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.3 for about 40% to about 100% of the perfume raw materials present in the perfume accord, for example, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.4 for about 35% to about 100% of the perfume raw materials present in the perfume accord, for example, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.5 for about 30% to about 100% of the perfume raw materials present in the perfume accord, for example, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.6 for about 25% to about 100% of the perfume raw materials present in the perfume accord, for example, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.7 for about 20% to about 100% of the perfume raw materials present in the perfume accord, for example, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/M is at least 0.8 for about 20% to about 100% of the perfume raw materials present in the perfume accord, for example, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 0.9 for about 20% to about 100% of the perfume raw materials present in the perfume accord, for example, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord. In some embodiments, Cᵢ/Mᵢ is at least 1.0 for about 15% to about 100% of the perfume raw materials present in the perfume accord, for example, about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, and/or about 95% to about 100% of the perfume raw materials present in the perfume accord.

The compositions disclosed herein have a high OIS value, such as an OIS value of at least about 2.0, at least about 3.0, at least about 4.0, at least about 5.0, at least about 5.5, at least about 6.0, at least about 7.0, at least about 7.5, at least about 8.0, at least about 9.0, at least about 10.0, at least about 12.0, at least about 14.0, at least about 16.0, at least about 18.0, at least about 20.0, at least about 22.0, at least about 24.0, at least about 26.0, at least about 28.0, and/or at least about 30.0. In some embodiments the compositions have a relatively higher OIS, such as about 7.5 to about 30.0, about 8.0 to about 28.0, about 10.0 to about 26.0, about 15.0 to about 24.0, and/or about 18.0 to about 22.0. In some embodiments the compositions have a relatively lower OIS, such as about 2.0 to about 10.0, about 4.0 to about 10.0, about 5.0 to about 9.0 and/or about 6.0 to about 8.0.

### Compositions

The leave-on compositions can have a low or very low concentration of a perfume accord and a high perceived odor intensity. Such compositions can include deodorant or antiperspirant compositions having an OIS of at least about 2.0, alternatively at least about 5.5 alternatively about 5.5 to about 20, or alternatively an OIS of about 6.0 to about 15.

In addition to a perfume accord, deodorant and antiperspirant compositions may include one or more silicones, active ingredients, odor blockers, optional ingredients, and/or ingredients specific to the product form. Other ingredients may be selected based on the product form and properties the formulator is trying to achieve.

### Silicones

The blend of a nonvolatile, low viscosity silicone emollient and high molecular weight silicone polymer may be present from about 3% to about 20% of the composition by weight, alternatively from about 5% to about 20% of the composition, alternatively from about 8% to about 15% of the composition, alternatively from about 10% to about 15% of the composition by weight, where the composition is in a solid, semi-solid, or aerosol form (excluding the propellant). The blend of a nonvolatile, low viscosity silicone emollient and high molecular weight silicone polymer may be present from about 10% to about 40% of the oil-phase of the composition, alternatively from about 15% to about 30% of the oil phase of the composition, where the composition is a gel or a roll-on.

The blend of a nonvolatile low viscosity silicone emollient and high molecular weight silicone polymer, such as dimethicone and dimethiconol, may be created prior to or during the product making process. Pre-blending is often preferred to allow the high molecular weight silicone polymer to be fully solvated by the low viscosity silicone emollient. The blend of a nonvolatile low viscosity silicone emollient and high molecular weight silicone polymer, such as dimethicone and dimethiconol, typically will have less than about 30% by weight of high molecular weight silicone polymer. It is preferred to have from about 5% to about 20% high molecular weight silicone polymer in the blend to create a blend with a viscosity that is easily incorporated into the product and more preferred to have from about 10% to about 15% high molecular weight silicone polymer in the blend. The high molecular weight silicone polymer may have a molecular weight greater than about 100 kilodaltons, or in some embodiments greater than about 300 kilodaltons, or in some other embodiments greater than about 500 kilodaltons. The nonvolatile low viscosity silicone emollient may have a viscosity less than about 100 cps and a vapor pressure less than about 0.10 mm of Hg.

Suitable commercial blends of a nonvolatile, low viscosity silicone emollient and a high molecular weight silicone polymer include, but are not limited to, DC1503, DC1413, and CB-1556 from Dow Chemical, Silsoft 8812 and SF1236 from Momentive Silicones, SPI-5403B from Silicone Plus, X-21-5613 from Shinetsu silicones, and Mirasil D-DML HV from Elkem Silicones.

Suitable high molecular weight silicone polymers may include, but are not limited to, X-21-5847 and X-21-5849 from Shinetsu silicones, Mirasil DM 500000 silicone and Mirasil DM 300 000 silicone from Elkem Silicones.

Suitable nonvolatile, low viscosity silicone emollients may include, but are not limited to, linear dimethicone emollients with a viscosity from about 5 cst about 100 cst. Other low viscosity silicone solvents such as phenyl trimethicone may also be employed.

In some embodiments, the nonvolatile, low viscosity silicone emollient may be a blend of multiple nonvolatile, low viscosity silicone emollients. Similarly, in some embodiments, the high molecular weight silicone polymer may be a mixture of multiple high molecular weight silicone polymers.

### Actives

The product composition may be an antiperspirant or deodorant composition. Antiperspirant products reduce sweating or perspiration on the skin, typically by temporarily blocking the pores and reducing the amount of sweat that reaches the surface. Deodorants work by combating the bacteria on the skin that break down sweat, which is what causes body odor. Antiperspirants can include deodorant actives and both antiperspirants and deodorants can include fragrances to mask and improve body odor.

If the product composition is an emulsion, the active is generally dissolved in the water phase. The concentration of the active in the composition should be sufficient to provide the finished antiperspirant or deodorant composition with the desired perspiration wetness and/or odor control benefits.

Exemplary antiperspirant active concentrations range include from about 0.1% to about 26%, from about 1% to about 20%, and from about 2% to about 10%, by weight of the composition. All such weight percentages are calculated on an anhydrous metal salt basis exclusive of water and any complexing or buffering agent such as, for example, glycine, glycine salts or other amino acids and any stabilizing agents such as calcium chloride, calcium salts, sodium salts or strontium salts.

Aluminum salts can have the general formula Al₂(OH)₆₋ₐXₐ wherein X is Cl, Br, I, or NO₃, and a is about 0.3 to about 5, alternatively from about 0.8 to about 2.5. Preferred actives in this group include, but are not limited to, aluminum chlorohydrate (ACH) wherein a is from about 1 and the mole ratio of Al/Cl is from about 1.9 to about 2.1, Aluminum sesquichlorohydrate (ASCH) wherein a is from about 1.05 to about 1.61 and the mole ratio of Al/Cl is from about 1.26 to about 1.89, and aluminum dichlorohydrate (ADCH) wherein a is from about 1.6 to about 2.2 and the mole ratio of Al/Cl is from about 0.9 to about 1.25.

The ACH salts may also include soluble calcium salts. Soluble calcium salts are those calcium salts that are soluble in water or that dissolve in the aqueous solution of antiperspirant salt (i.e., a solution of the aluminum salt and/or zirconium salt). Calcium salts which may be utilized are any of those which do not otherwise interfere with the solubility or effectiveness of the antiperspirant salt. Preferred calcium salts include calcium chloride, calcium bromide, calcium nitrate, calcium citrate, calcium formate, calcium acetate, calcium gluconate, calcium ascorbate, calcium lactate, calcium glycinate and mixtures thereof. Calcium carbonate, calcium sulfate and calcium hydroxide may also be used because they will dissolve in an aqueous solution of the antiperspirant salt.

The ACH salts may also include soluble sodium salts. Soluble sodium salts are those sodium salts that are soluble in water or that dissolve in the aqueous solution of antiperspirant salt (i.e., a solution of the aluminum salt and/or zirconium salt). Sodium salts which may be utilized are any of those which do not otherwise interfere with the solubility or effectiveness of the antiperspirant salt. Sodium salts can include sodium chloride, sodium nitrate, and mixtures thereof. Sodium sulfate may also be used because they will dissolve in an aqueous solution of the antiperspirant salt.

The ACH salt can be chosen from sodium chloride, potassium chloride, lithium chloride, or mixtures thereof.

Additional ACH actives are disclosed in U.S. patent application Ser. No. 19/349,063, incorporated by reference.

The ACH salts used may also contain a water soluble amino and/or hydroxy acid which is effective in increasing and/or stabilizing the HPLC peak 4:3 area ratio of the antiperspirant salt.

Such acids include amino- and/or hydroxy-substituted lower alkanoic acids (including substituted derivatives thereof), preferably where the amino or hydroxy group is located on the α-carbon (i.e., the same carbon to which the carboxy group is attached). The lower alkanoic acid will generally have 2 to 6, preferably 2 to 4, carbon atoms in the alkanoic acid chain. Typical amino and/or hydroxy substituted lower alkanoic acids include any of the amino acids such as glycine, alanine, valine, leucine, isoleucine, P-alanine, serine, cysteine, β-amino-n-butyric acid, γ-amino-n-butyric acid, etc. and hydroxy acids such as glycolic acid and lactic acid. These amino and/or hydroxy substituted lower alkanoic acids may also contain various substituents which do not adversely affect their activity. The preferred amino and/or hydroxy substituted lower alkanoic acids are glycine, alanine, and glycolic acid, with glycine being most preferred. The active can include an aqueous solution of ACH that contains calcium chloride, sodium chloride and glycine. The ACH can have a molar ratio of glycine to calcium of less than 2, alternatively less than or equal to 1.75, alternatively less than or equal to 1.5. The ACH can have a pH of about 4.2 to about 5.3, alternatively from about 4.5 to about 5.0. The ACH can have a peak IV/III ratio exceeding 0.75 as measured by HPLC.

The active can be an aqueous solution of ADCH that also contains calcium chloride and glycine. The preferred ADCH with calcium chloride and glycine is further characterized by having more than 50% peak 4 and 5 as measured by HPLC, a Al:Cl molar ratio of about 0.9 to about 1.25, an Al to glycine wt. ratio of about 1.7 to 7.7, and calcium to glycine wt. ratio of about 0.1 to about 1.5. The active can be an aqueous solution of ASCH that also contains calcium chloride and glycine. The preferred ASCH with calcium chloride and glycine is further characterized by having more than 35% peak 4 and 5 as measured by HPLC, a Al:Cl molar ratio of about 1.26 to about 1.89, an Al to glycine wt ratio of about 4 to 10, and calcium to glycine ratio of about 0.1 to about 1.5.

The antiperspirant or deodorant compositions provided herein may comprise a non-aluminum antiperspirant active. Suitable non-aluminum antiperspirant actives include, but are not limited to, oxybutynin chloride, chitosan, PVM/MA polymers, calcium channel blockers, gingerol, liquid fatty acid and metal ion combinations, magnesium gluconate, silicic acid, silicic acid salts, and vicinal diols such as propylene glycol.

The compositions may comprise a deodorant active. The deodorant active can be used instead of an antiperspirant active or in addition an antiperspirant active. Some deodorants may not have an antiperspirant active and/or may be substantially free or free of aluminum. Suitable deodorant actives may be chosen from antimicrobial agents (e.g., bactericides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctone, especially zinc salts, and acids thereof, heavy metal salts of pyrithione, especially zinc pyrithione, zinc phenolsulfate, farnesol, and combinations thereof.

The deodorant active may include an antibacterial chosen from 2-Pyridinol-N-oxide (piroctone olamine), lupamin, beryllium carbonate, magnesium carbonate, calcium carbonate, magnesium hydroxide, magnesium hydroxide and magnesium carbonate hydroxide, partially carbonated magnesium hydroxide, magnesium oxide, potassium carbonate, potassium bicarbonate, sodium carbonate, sodium sesquicarbonate, baking soda, hexamidine, zinc carbonate, thymol, polyvinyl formate, salicylic acid, niacinamide and combinations thereof.

The concentration of the deodorant active may range, individually or cumulatively, from about 0.001%, from about 0.01%, of from about 0.1%, by weight of the composition to about 20%, to about 10%, to about 5%, or to about 1%, by weight of the composition.

### Odor Entrappers

The composition can include an odor entrapper. Suitable odor entrappers can include solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms; therefore, this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

### Optional Ingredients

The compositions can further comprise optional ingredients that can include colorants, cosmetic powders, chelants, preservatives, vitamins including vitamin A (retinol), vitamin B3 (niacinamide), vitamin B5 (panthenol), vitamin B7 (biotin), vitamin C (ascorbic acid), vitamin D, vitamin E, vitamin K, hyaluronic acid, collagen, peptides, and Omega fatty acids. Examples of such optional materials are described in U.S. Pat. No. 4,049,792; Canadian Patent 1,164,347; U.S. Pat. Nos. 5,019,375; and 5,429,816; which descriptions are incorporated herein by reference.

When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Fragrance Allergens

The leave-on compositions can include less than or equal to 0.001% of a fragrance allergen chosen from benzyl alcohol, 6-methyl coumarin, citral, isoeugenol, citronellol, limonene, Pinus mugo, Pinus pumila, Cedrus atlantica oil/extract, turpentine, alpha-terpinene, terpinolene, myroxylon pereirae oil/extract, rose ketones, 3-propylidenephthalide, Lippia citriodora absolute, methyl salicylate, acetyl cedrene, amyl salicylate, anethole, benzaldehyde, camphor, beta-caryophyllene, carvone, dimethyl phenethyl acetate, hexadecanolactone, hexamethylindanopyran, linalyl acetate, menthol, trimethylcyclopentenyl methylisopentenol, salicylaldehyde, santalol, sclareol, terpineol, tetramethyl acetyloctahydronaphthalenes, trimethylbenzenepropanol, vanillin, Cananga odorata oil/extract, Cinnamomum cassia leaf oil, Cinnamomum zeylanicum bark oil, Citrus aurantium flower oil, Citrus aurantium peel oil, Citrus aurantium bergamia peel oil, Citrus limon peel oil, lemongrass oil, Eucalyptus globulus oil, Eugenia caryophyllus oil, jasmine oil/extract, Juniperus virginiana oil, Laurus nobilis leaf oil, lavandula oil/extract, Mentha piperita oil, Mentha viridis leaf oil (spearmint oil), narcissus extract, Pelargonium graveolens flower oil, Pogostemon cablin oil, rose flower oil/extract, santalum album oil (sandalwood oil), eugenyl acetate, geranyl acetate, isoeugenyl acetate, and pinene, or combinations thereof.

### Product Forms

Antiperspirant and deodorant compositions can be formulated in many forms. For example, an antiperspirant or deodorant composition can be, without limitation, a roll-on product, a gel, a stick including soft solid sticks, invisible solids, glycol sticks, or sticks made from natural waxes with or without silicones, or an aerosol including both deodorant or antiperspirant sprays with hydrocarbon and/or compressed gas propellants.

### Roll-On and Gel

A roll-on or gel antiperspirant or deodorant composition can comprise, for example, water, emollient, emulsifiers, deodorant or antiperspirant actives, or combinations thereof.

### Water

The roll-on or gel composition can include water. Water can be present in an amount of about 1% to about 99.5%, about 25% to about 99.5%, about 50% to about 99.5%, about 75% to about 99.5% about 80% to about 99.5%, from about 20% to about 60%, from about 40% to about 70% or any combination of the end points and points encompassed within the ranges, by weight of the deodorant composition.

### Emollients

Roll-on or gel compositions can comprise an emollient system including at least one emollient but may also comprise a combination of emollients. Suitable emollients are often liquid under ambient conditions. Depending on the type of product form desired, concentrations of the emollient(s) in the deodorant or antiperspirant compositions can range from about 1% to about 95%, from about 5% to about 95%, from about 15% to about 75%, from about 1% to about 10%, from about 15% to about 45%, or from about 1% to about 30%, by weight of the deodorant or antiperspirant composition.

Emollients suitable for use in the roll-on and gel compositions include, but are not limited to, propylene glycol, polypropylene glycol (like dipropylene glycol, tripropylene glycol, etc.), diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, alkyl ethers of polyhydric and monohydric alcohols, volatile silicone emollients such as cyclopentasiloxane, nonvolatile silicone emollients such as dimethicone, mineral oils, polydecenes, petrolatum, and combinations thereof. One example of a suitable emollient comprises for roll-ons is PPG-15 stearyl ether. Examples of suitable emollients for a gel are cyclopentasiloxane and dimethicone. Other examples of suitable emollients or both forms include dipropylene glycol and propylene glycol.

### Emulsifier

The composition can contain an emulsifier. A suitable emulsifier can be, for example, a polymeric or nonpolymeric surfactants. Suitable surfactants are nonionic surfactants, cationic surfactants, amphoteric surfactants, zwitterionic surfactants, and mixtures thereof. Often roll-ons employ emulsifiers suitable for oil in water emulsions. Examples of emulsifiers for gel compositions can include those suitable for water in silicone or water in oil emulsions.

Suitable emulsifiers for roll-on products can include, for example, polyoxyethylene 2 stearyl ether, polyoxyethylene 20 stearyl ether, polyoxyethylene 21 stearyl ether, ceteth-10, ceteareth-2, and combinations thereof. Suitable emulsifiers for gel compositions can include but are not limited to IEG/PPG-18/18 Dimethicone (DC5225), Lauryl PEG/PPG-18/18 methicone, Dimethicone/PEG-10/15 Crosspolymer, and combinations thereof.

When the emulsifier is present, it is typically present at a level of from about 0.01% to about 10%, alternatively from about 0.01% to about 3%, alternatively from about 0.05% to about 1%, alternatively from about 0.01% to about 0.05%, by weight of the composition.

### Stick Products

Stick products including solid, invisible solid, and soft solid antiperspirant or deodorant compositions as described herein can contain a primary structurant and sometimes additional structurants, emollients, an antiperspirant and/or deodorant active, a perfume, perfume delivery composition, and additional chassis ingredient(s). These antiperspirants or deodorant composition may be anhydrous. The antiperspirant or deodorant stick composition may be free of added water.

### Hardness

The compositions can be in the form of a solid stick including invisible solids. The stick compositions can have a product hardness of about 600 gram·force or more. The solid or invisible solid can have a product hardness of least about 600 gram·force, more specifically from about 600 gram·force to about 5,000 gram·force, still more specifically from about 750 gram·force to about 2,000 gram·force, and yet more specifically from about 800 gram·force to about 1,400 gram·force. Moreover, the compositions can form a soft solid that is often rubbed onto the skin via a package with an application surface. The soft solid stick compositions can have a product hardness of less than about 600 gram·force, specifically from about 100 to 500 gram·force.

The term “product hardness” or “hardness” as used herein is a reflection of how much force is required to move a penetration cone a specified distance and at a controlled rate into an antiperspirant composition under the test conditions described herein below. Higher values represent harder product, and lower values represent softer product. These values are measured at 27 °C, 15% relative humidity, using a TA-XT2 Texture Analyzer, available from Texture Technology Corp., Scarsdale, N.Y., U.S.A. The product hardness value as used herein represents the peak force required to move a standard 45-degree angle penetration cone through the composition for a distance of 10 mm at a speed of 2 mm/second. The standard cone is available from Texture Technology Corp., as part number TA-15, and has a total cone length of about 24.7 mm, angled cone length of about 18.3 mm, and a maximum diameter of the angled surface of the cone of about 15.5 mm. The cone is a smooth, stainless steel construction and weighs about 17.8 grams.

### Structurants

The stick formulation can comprise a suitable concentration of a primary structurant to help provide the antiperspirant with the desired viscosity, rheology, texture and/or product hardness, or to otherwise help suspend any dispersed solids or liquids within the composition.

The term “solid structurant” as used herein means any material known or otherwise effective in providing suspending, gelling, viscosifying, solidifying, and/or thickening properties to the composition or which otherwise provide structure to the final product form. These solid structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof.

The concentration and type of solid structurant selected for use in the antiperspirant or deodorant compositions will vary depending upon the desired product hardness, rheology, and/or other related product characteristics. For most structurants suitable for use herein, the total structurant concentration ranges from about 5% to about 35%, more typically from about 10% to about 30%, or from about 7% to about 20%, by weight of the composition.

Non-limiting examples of suitable structurants can include stearyl alcohol, cetyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fischer-Tropsch waxes, and microcrystalline wax; polyethylenes with molecular weight of 200 to 1000 daltons; solid triglycerides such as tribehenin and C18-C36 triglyceride; behenyl alcohol, or combinations thereof.

Other non-limiting examples of structurants suitable for use herein are described in U.S. Pat. Nos. 5,976,514; 5,891,424; and 10,966,915 the descriptions of which are incorporated herein by reference.

### Emollients

The stick composition can comprise an emollient or mixture of emollients at concentrations ranging from about 20% to about 80%, and more specifically from about 30% to about 70%, by weight of the composition. The emollient can be a volatile silicone which may be cyclic or linear. The emollient can a be a nonvolatile silicone that is linear.

“Volatile silicone” as used herein refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976), which descriptions are incorporated herein by reference.

The volatile silicone can be a cyclic silicone having from 3 to 7, and more specifically from 5 to 6, silicon atoms, and still more specifically 5, like cyclopentasiloxane. These cyclic silicone materials will generally have viscosities of less than about 10 centistokes at 25 °C.

Linear volatile silicone materials suitable for use in the antiperspirant compositions can include those represented by the formula:

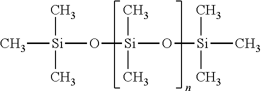

wherein n is from 1 to 7, and more specifically from 2 to 3. These linear silicone materials will generally have viscosities of less than about 5 centistokes at 25 °C.

Specific examples of volatile silicone solvents suitable for use in the antiperspirant compositions can include, but are not limited to, Cyclomethicone D-5; GE 7207 and GE 7158 (commercially available from General Electric Co.); Dow Corning 344; Dow Corning 345; Dow Corning 200; and DC1184 (commercially available from Dow Corning Corp.); and SWS-03314 (commercially available from SWS Silicones).

### Nonvolatile Emollient

Nonvolatile emollients may be also present in these compositions. Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate, and isobutyl stearate. The nonvolatile emollient could also comprise a silicone emollient such as dimethicone with a viscosity of 5 cst or greater.

### C. Aerosols

Aerosol products can include antiperspirant sprays and/or deodorant body sprays. Both forms may contain a propellant and a concentrate. The product concentrate may contain, for example a carrier liquid, an antiperspirant or deodorant active, and a perfume.

### Propellant

The aerosol compositions described herein can include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 90%, 85% 80% 70%, 65%, 60%, 54%, 52%, 50%, 48%, 46%, 44%, or 42%, or any combination thereof, by weight of the total fill of materials stored within the container.

The propellant can be a compressed gas, which can be in the gas state at 25 °C and at a pressure of at least 50 psi, such as compressed air, oxygen, nitrogen, carbon dioxide, or mixtures thereof. The composition can include from about 0.5% to about 25% compressed gas, from about 1% to about 15%, from about 2% to about 12%, from about 3% to about 10%, from about 3% to about 7%, and from about 4% to about 5%.

### Carrier

A carrier liquid suitable for use in a deodorant body spray can include water, alcohol, or combinations thereof. The carrier may be present in an amount of about 1% to about 99.5%, about 25% to about 99.5%, about 50% to about 99.5%, about 75% to about 99.5% about 80% to about 99.5%, from about 15% to about 45%, or any combination of the end points and points encompassed within the ranges, by weight of the composition. A suitable example of an alcohol can include ethanol. Often ethanol is the predominate carrier liquid in a deodorant body spray. The aerosol product can be anhydrous.

Carrier liquids suitable for antiperspirant concentrates can include an oil or a mixture of two or more oils. The carrier may be present in an amount of about 1% to about 99.5%, about 25% to about 99.5%, about 50% to about 99.5%, about 75% to about 99.5% about 80% to about 99.5%, from about 15% to about 45%, or any combination of the end points and points encompassed within the ranges, by weight of the composition. Useful oils include, for example, volatile silicone oils and non-volatile organic oils. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976). The volatile silicone can be a cyclic silicone having from at least about 3 silicone atoms or from at least about 5 silicone atoms but no more than about 7 silicone atoms or no more than about 6 silicone atoms. For example, volatile silicones can be used which conform to the formula:

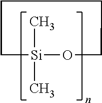

wherein n is from about 3 or from about 5 but no more than about 7 or no more than about 6. These volatile cyclic silicones generally have a viscosity of less than about 10 centistokes at 25 °C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); MASIL SF-V (available from Mazer) and combinations thereof. Suitable volatile silicone oils can also include linear silicone oils such as, for example, DC200 (1 cSt), DC200 (0.65 cSt), and DC2-1184, all of which are available from Dow Corning Corp. In certain examples, the volatile silicone oil can have a viscosity of less than 10 centistokes at 25 °C.

Non-volatile organic, emollient oils can also be employed. A representative, non-limiting list of emollient oils includes CETIOL CC (dicaprylyl carbonate), CETIOL OE (dicaprylyl ether), CETIOL S (diethylhexylcyclohexane), and CETIOL B (dibutyl adipate), all of which are available from Cognis, and LEXFEEL 7 (neopentyl glycol diheptanoate) from Inolex. In certain examples, the organic emollient oils have a viscosity of less than 50 centistokes at 25 °C. The term “organic emollient oil” as used herein means silicon-free emollient oils that are liquid at 25 °C, and that are safe and light to skin and can be miscible with volatile silicone oils (as described above).

### Cream

The composition can be a cream that includes a moisturizer, for instance a moisturizer specifically designed for use on the face or body, as well as a whole-body deodorant cream designed for underarms, privates, chest including underboobs, hands, feet, etc. The cream can be combined with sunscreen actives.

The cream can include a dermatologically acceptable carrier. The carrier can be present at a level of from about 50% to about 99%, about 60% to about 98%, about 70% to about 98%, or, alternatively, from about 80% to about 95%, by weight of the composition.

The cream can have a pH of less than about 4.0, and typically greater than 1.0.

Non-limiting examples include simple solutions (e.g., aqueous or anhydrous), dispersions, emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil or oil-in-water). The oil phase may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). Emulsions may also contain an emulsifier, e.g., from about 1% to about 10% or from about 2% to about 5% based on the weight of the carrier. Emulsifiers may be nonionic, anionic or cationic. Some suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986), each incorporated herein by reference.

The carrier may contain one or more dermatologically acceptable, hydrophilic diluents. As used herein “diluent” includes materials in which the niacinamide and/or saccharide can be dispersed, dissolved, or otherwise incorporated. Hydrophilic diluents include water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Thickeners

In some instances, it may be desirable to use thickeners, in particular ones that tolerate a lower range of pH. For example, neutralized thickeners may not impart the desired thickening or feel properties to the composition at low pH. On the other hand, fatty alcohols such as cetyl alcohol and stearyl alcohol are generally stable at low pH, and thus particularly suited for use in the low pH compositions herein. Accordingly, the present compositions may be free or substantially free of neutralized thickeners and/or may have from 0.1% to 10% (e.g., from about 0.5% to about 8%, from about 1.0% to about 5%, or even from about 2% to about 4%) of a fatty alcohol thickener.

U.S. Pat. No. 9,833,398 includes additional examples and materials that can be incorporated into skin care compositions and is incorporated by reference herein.

### DEFINITIONS

The term “substantially free of” refers to an amount of a material that is less than 1%, 0.5%, 0.25%, 0.1%, 0.05%, 0.01%, or 0.001% by weight of an antiperspirant composition. “Free of” refers to no detectable amount of the stated ingredient or thing.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21 °C and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### EXAMPLES

Comparative perfume accords were prepared according to the formulations provided in Table 7 and Table 8. The amount of perfume raw material provided in the table is a percent by weight (wt. %) based on the total weight of the perfume accord (i.e., the blend of perfume raw materials).

Table 7 lists the wt. % of each perfume raw material in Perfume Accord A, M for each perfume raw material, and C/M for each perfume raw material when 1.0% Perfume Accord A is present in the composition. Perfume Accord A demonstrates an odor intensity score (01S) in a leave-on composition of an OIS of 11.0, as obtained by summing all C/M values in the table and dividing by the total number of perfume raw materials.

TABLE 7
Perfume Accord A

| Material Name | wt % | M, ppm | C/M |
|---|---|---|---|
| ethyl 2-methylbutanoate | 2 | 6.46 | 30.9421 |
| octanal | 0.2 | 46.08 | 0.4340 |
| ethyl hexanoate | 0.2 | 9.71 | 2.0592 |
| allyl 3-cyclohexylpropanoate | 1 | 72.30 | 1.3832 |
| (Z)-hex-3-en-1-yl acetate | 0.5 | 9.99 | 5.0060 |
| decanal | 0.2 | 31.68 | 0.6313 |
| 2,6-dimethyloct-7-en-2-ol | 1.6 | 74.62 | 2.1441 |
| benzyl acetate | 1.45 | 101.43 | 1.4296 |
| (Z)-hex-3-en-1-yl butyrate | 0.5 | 51.27 | 0.9752 |
| 4-(tert-butyl)cyclohexyl acetate | 5 | 159.33 | 3.1382 |
| Ethyl (1R,6S)-2,2,6- | 0.4 | 66.07 | 0.6054 |
| trimethylcyclohexane-1- | | | |
| carboxylate | | | |
| 5-butyldihydro-2(3H)-furanone | 12 | 21712.31 | 0.0553 |
| 3-(4-isopropylphenyl)propanal | 1 | 63.76 | 1.5683 |
| (3R,3aR,6S,7S,8aS)-6-methoxy- | 1 | 1825.70 | 0.0548 |
| 3,6,8,8-tetramethyloctahydro-1H- | | | |
| 3a,7-methanoazulene | | | |
| (3aR,5aS,9aS,9bR)-3a,6,6,9a- | 2 | 452.85 | 0.4417 |
| tetramethyl-2,4,5,5a,7,8,9,9b- | | | |
| octahydro-1H- | | | |
| benzo[e][1]benzofuran | | | |
| 5-pentyldihydrofuran-2(3H)-one | 13 | 165.01 | 7.8785 |
| 6-pentyltetrahydro-2H-pyran-2-one | 2 | 2085.58 | 0.0959 |
| octahydro-2H-chromen-2-one | 7 | 79775.22 | 0.0088 |
| 3-butyl-5-methyltetrahydro-2H- | 5 | 1797.61 | 0.2781 |
| pyran-4-yl acetate | | | |
| 3,4-dimethoxybenzaldehyde | 1 | 330042.26 | 0.0003 |
| 7-methyl-4,4a,5,6- | 1 | 188781.47 | 0.0005 |
| tetrahydronaphthalen-2(3H)-one | | | |
| 2-(4-methylthiazol-5-yl)ethan-1-ol | 0.05 | 375.85 | 0.0133 |
| 6-heptyltetrahydro-2H-pyran-2-one | 4 | 4.84 | 82.5711 |
| (1-methyl-2-((1,2,2- | 10 | 6.36 | 157.1862 |
| trimethylbicyclo(3.1.0)hexan-3- | | | |
| yl)methyl)cyclopropyl)methanol | | | |
| 3-ethoxy-4-hydroxybenzaldehyde | 6 | 124.74 | 4.8099 |
| 4-formyl-2-methoxyphenyl | 0.8 | 141568.50 | 0.0006 |
| isobutyrate | | | |
| oxacyclohexadecan-2-one | 5 | 5189.70 | 0.0963 |
| (E)-3-methylcyclopentadec-4-en-1- | 5 | 34.91 | 14.3235 |
| one | | | |
| (E)-oxacyclohexadec-13-en-2-one | 11.1 | 18300.10 | 0.0607 |

Table 8 lists the wt. M of each perfume raw material in Perfume Accord B, M for each perfume raw material, and C/M for each perfume raw material when 1.01 Perfume Accord B is present in the composition. Perfume Accord B demonstrates an odor intensity score (GIS) in a leave-on composition of an OIS of 5.2, which is less than half of the OIS for Perfume Accord A. Therefore, an efficient composition comprising Perfume Accord A demonstrated a significantly higher OIS relative to the comparative compositions comprising Perfume Accord B when present at the same concentration.

TABLE 8
Perfume Accord B

| Material Name | wt % | M, ppm | C/M |
|---|---|---|---|
| ethyl butyrate | 0.024 | 8.23 | 0.2918 |
| ethyl 2-methylpentanoate | 0.084 | 9.98 | 0.8414 |
| octanal | 0.057 | 46.08 | 0.1237 |
| 1,3,3-trimethyl-2- | 0.91 | 8.00 | 11.3767 |
| oxabicyclo(2.2.2)octane | | | |
| 1-methyl-4-(prop-1-en-2- | 3.235 | 553.65 | 0.5843 |
| yl)cyclohex-1-ene | | | |
| hexyl acetate | 0.17 | 23.44 | 0.7252 |
| 6-methylhept-5-en-2-one | 0.12 | 455.69 | 0.0263 |
| allyl 3-cyclohexylpropanoate | 0.14 | 72.30 | 0.1936 |
| (Z)-hex-3-en-1-yl acetate | 0.012 | 9.99 | 0.1201 |
| 1-methyl-4-(propan-2- | 0.019 | 386.52 | 0.0049 |
| ylidene)cyclohex-1-ene | | | |
| (Z)-hex-3-en-1-ol | 1 | 75.00 | 1.3333 |
| benzaldehyde | 0.011 | 114.10 | 0.0096 |
| 2,6-dimethylhept-5-enal | 0.7 | 9.48 | 7.3872 |
| 2,4-dimethylcyclohex-3-ene-1- | 2 | 16.82 | 11.8899 |
| carbaldehyde | | | |
| (2S,5R)-2-isopropyl-5- | 0.76 | 2.06 | 36.9255 |
| methylcyclohexan-1-one | | | |
| 1,7,7- | 0.66 | 10.02 | 6.5874 |
| trimethylbicyclo(2.2.1)heptan-2- | | | |
| one | | | |
| decanal | 0.46 | 31.68 | 1.4519 |
| 2,6-dimethyloct-7-en-2-ol | 21.783 | 74.62 | 29.1912 |
| 3,7-dimethylocta-1,6-dien-3-yl | 0.39 | 382.25 | 0.1020 |
| acetate | | | |
| 2-(tert-butyl)cyclohexyl acetate | 2.6 | 28.14 | 9.2405 |
| 3,7-dimethylocta-1,6-dien-3-ol | 6.2 | 34.48 | 17.9824 |
| (E)-3,7-dimethylocta-2,6-dienal | 0.42 | 6.45 | 6.5124 |
| 2-[(1R)-4-methylcyclohex-3-en-1- | 3.084 | 512.12 | 0.6022 |
| yl]propan-2-yl acetate | | | |
| 2-isopropyl-5-methylphenol | 0.37 | 68036.31 | 0.0005 |
| (1R,2S,5R)-2-isopropyl-5- | 0.86 | 881.68 | 0.0975 |
| methylcyclohexan-1-ol | | | |
| 3,7-dimethyloct-6-enenitrile | 2.6 | 35.56 | 7.3113 |
| (E)-3,7-dimethylocta-2,6-dien-1- | 0.14 | 12.79 | 1.0948 |
| ol | | | |
| (Z)-3,7-dimethylocta-2,6-dien-1- | 0.076 | 85.25 | 0.0891 |
| ol | | | |
| (1R,9S,Z)-4,11,11-trimethyl-8- | 0.92 | 148.90 | 0.6179 |
| methylenebicyclo(7.2.0)undec-4- | | | |
| ene | | | |
| 4-allyl-2-methoxyphenol | 1 | 68.51 | 1.4597 |
| (3aR,5aS,9aS,9bR)-3a,6,6,9a- | 0.33 | 452.85 | 0.0729 |
| tetramethyl-2,4,5,5a,7,8,9,9b- | | | |
| octahydro-1H- | | | |
| benzo[e][1]benzofuran | | | |
| 2-methylundecanal | 0.048 | 11.36 | 0.4225 |
| 3-(4-isopropylphenyl)-2- | 5.7 | 382.77 | 1.4891 |
| methylpropanal | | | |
| (Z)-1-(2,6,6-trimethylcyclohex-2- | 0.02 | 23.65 | 0.0846 |
| en-1-y1)but-2-en-1-one | | | |
| 3-(2-ethylphenyl)-2,2- | 0.98 | 155.15 | 0.6316 |
| dimethylpropanal | | | |
| 6-(sec-butyl)quinoline | 0.27 | 22.29 | 1.2111 |
| (Z)-4-(6,6-dimethylcyclohex-2- | 0.11 | 6.30 | 1.7451 |
| en-1-yl)-3-methylbut-3-en-2-one | | | |
| 4-(4-Methyl-3-pentenyl)-3- | 1 | 4636.02 | 0.0216 |
| cyclohexene-1-carboxaldehyde | | | |
| (Z)-3-methyl-4-(2,6,6- | 0.95 | 3.15 | 30.1261 |
| trimethylcyclohex-2-en-1-yl)but- | | | |
| 3-en-2-one | | | |
| 6-heptyltetrahydro-2H-pyran-2- | 0.41 | 4.84 | 8.4635 |
| one | | | |
| pentyl 2-hydroxybenzoate | 0.11 | 759.48 | 0.0145 |
| 2H-chromen-2-one | 1.4 | 1224.90 | 0.1143 |
| 1-(naphthalen-2-yl)ethan-1-one | 2.3 | 6989.85 | 0.0329 |
| 7-methyl-2H- | 0.088 | 2156.94 | 0.0041 |
| benzo(b)(1,4)dioxepin-3(4H)-one | | | |
| (Z)-2-benzylideneoctanal | 15 | 24.66 | 60.8301 |
| 1-((2S,3S)-2,3,8,8-tetramethyl- | 14 | 522.84 | 2.6777 |
| 1,2,3,4,5,6,7,8- | | | |
| octahydronaphthalen-2-yl)ethan- | | | |
| 1-one | | | |
| hexyl 2-hydroxybenzoate | 0.44 | 4109.92 | 0.0107 |
| 1-(2,2,6- | 1.7 | 46.86 | 3.6276 |
| trimethylcyclohexyl)hexan-3-ol | | | |
| (Z)-2-ethyl-4-(2,2,3- | 1.4 | 234.79 | 0.5963 |
| trimethylcyclopent-3-en-1-yl)but- | | | |
| 2-en-1-ol | | | |
| oxacyclohexadecan-2-one | 2.5 | 5189.70 | 0.0482 |
| (E)-3-methylcyclopentadec-4-en- | 0.12 | 34.91 | 0.3438 |
| 1-one | | | |

The following examples are given solely for the purpose of illustration and are not to be construed as limitations, as many variations thereof are possible.

TABLE 9
Invisible Solid Example

| | Purpose | (wt. %) |
|---|---|---|
| Aluminum Zirconium Trichlorohydrex | Antiperspirant active | 14.00 |
| Gly (19% Anhydrous) | | |
| Cyclopentasiloxane | Volatile emollient | Q.S. |
| Stearyl Alcohol | Structurant | 14.00 |
| C12-15 Alkyl Benzoate | Nonvolatile emollient | 9.50 |
| Ppg-14 Butyl Ether | Nonvolatile emollient | 6.50 |
| Hydrogenated Castor Oil | Structurant | 3.85 |
| Petrolatum | Nonvolatile emollient | 3.00 |
| Talc | | 2.10 |
| Phenyl Trimethicone | Nonvolatile emollient | 3.00 |
| Mineral Oil | Nonvolatile emollient | 1.00 |
| Behenyl Alcohol | Structurant | 0.20 |
| Dimethicone (and) Dimethiconol | | 10.00 |
| (12%) (commercially available as | | |
| DC1503 from Dow ®) | | |
| Perfume | | 2-2.5 |

TABLE 10
Soft Solid Formulation Example

| | Purpose | (wt. %) |
|---|---|---|
| Cyclopentasiloxane | Emollient | Q.S. |
| Aluminum Zirconium Trichlorohydrate | Antiperspirant Active | 25.25 |
| Gly Powder | | |
| Fully Hydrogenated High Erucic | Structurant | 5.00 |
| Rapeseed Oil | | |
| Dimethicone | Emollient | 5.00 |
| Petrolatum | Emollient | 3.00 |
| C18-36 Acid Triglyceride | Structurant | 1.25 |
| Beta Cyclodextrin with Perfume | Fragrance delivery | 3.00 |
| | system | |
| Perfume | | 2-2.5 |
| Dimethicone (and) Dimethiconol | Emollient | 5 |
| (12%) (commercially available as | | |
| DC1503 from Dow ®) | | |

TABLE 11
Spray Formulation with Hydrocarbon Propellant Example

| | Purpose | (wt. %) |
|---|---|---|
| Butane, Propane, Isobutane | Propellant | 80.20 |
| Cyclopentasiloxane | Emollient | Q.S. |
| Aluminum Chlorohydrate | Antiperspirant Active | 5.00 |
| Disteardimonium Hectorite | Structurant | 0.60 |
| Dimethicone, 10 cst | Emollient | 0.50 |
| Triethyl Citrate | Emollient | 0.20 |
| Dimethicone (and) Dimethiconol | Emollient | 2 |
| (12%) (commercially available as | | |
| DC1503 from Dow ®) | | |
| Perfume | | 1.5-2 |

TABLE 12
Spray Formulation with Compressed Gas Propellant Examples

| | Ex. A | Ex. B | Ex. C | Ex. D |
|---|---|---|---|---|
| | (wt. %) | (wt. %) | (wt. %) | (wt. %) |
| Ethanol | Q.S. | | | 30 |
| Caprylic/Capric | 12.00 | | | 20 |
| Triglyceride | | | | |
| Dipropylene Glycol | 14.00 | 67.52 | 61.0 | |
| Propylene glycol | | 24.50 | | |
| Water | 5 | Q.S. | Q.S. | Q.S. |
| Citric Acid | | | 1.0 | 0.35 |
| Triethylcitrate | | | 1.0 | 1 |
| Piroctone Olamine | | 0.10 | 0.1 | 0.10 |
| Polysorbate 20 | | | | 0.30 |
| Sodium Magnesium | | | | 0.50 |
| Fluorosilicate | | | | |
| Xanthan Gum | | | | 0.05 |
| Perfume | 1.50 | 2 | 2 | 2 |
| Nitrogen Propellant | 1-5 | 1-5 | 1-5 | 1-5 |

TABLE 13
Roll-on Formulation Example

| | Purpose | (wt. %) |
|---|---|---|
| Aluminum Chlorohydrate | Antiperspirant | 14.40 |
| | Active | |
| Water | solvent | 75.95 |
| Ppg-15 Stearyl Ether | Emollient | 3.00 |
| Steareth-2 | Emulsifier | 1.90 |
| Steareth-20 | Emulsifier | 1.10 |
| Butylated Hydroxytoluene (BHT) | Antioxidant | 0.05 |
| Disodium EDTA | Chelant | 0.10 |
| Dimethicone (and) Dimethiconol (12%) | Emollient | 3.00 |
| (commercially available as DC1503 from | | |
| Dow ®) | | |
| Perfume | | 1 |

TABLE 14
Gel Formulation Example

| | | (wt. %) |
|---|---|---|
| Aluminum Zirconium Octachlorohydrex | Antiperspirant | 55.15 |
| Gly (29% anhydrous) | Active | |
| Water | Solvent | 10.95 |
| Alcohol Denat. | Solvent | 11.00 |
| Propylene Glycol | Emollient | 6.15 |
| Cyclopentasiloxane (and) PEG/PPG-18/18 | Emulsifier | 7.50 |
| (commercially available as DOWSIL ™ | | |
| 5225C from Dow ® | | |
| Dimethicone (and) Dimethiconol (12%) | Emollient | 5.00 |
| (commercially available as DC1503 from | | |
| Dow ®) | | |
| Cyclopentasiloxane | Emollient | 3.5 |
| Perfume | | 1 |

TABLE 15
Glycol Based Stick Examples

| | Ex. E | Ex. F | Ex. G | Ex. H | Ex. I |
|---|---|---|---|---|---|
| | (wt. %) | (wt. %) | (wt. %) | (wt. %) | (wt. %) |
| Dipropylene glycol | 25 | 18 | 17.8 | 17.8 | 17.8 |
| Propylene glycol | 10.1 | 7.15 | 7.07 | 7.07 | 7.07 |
| 1,3-propanediol | 22.5 | 30 | 30 | 30 | 30 |
| Glycerin | 7.5 | 10 | 10 | 10 | 10 |
| Water, deionized | QS | QS | QS | QS | QS |
| Sodium stearate (commercially | 4 | 4 | 4 | 4 | 3.5 |
| available as OP-200V, from | | | | | |
| Hallstar ®) | | | | | |
| Perfume | 3-4 | 3-4 | 3-4 | 3-4 | 3-4 |
| Minors including PPG-3-myristyl | 2.1 | 2.1 | 2.1 | 2.1 | 2.1 |
| ether, and tetrasodium EDTA | | | | | |
| Poloxamine 1307 | 3 | 3 | 3 | 3 | 3 |
| Antioxidant (INCI: Octadecyl | 0.03 | 0.03 | 0.28 | 0.28 | 0.28 |
| Di-t-butyl-4-hydroxyhydrocinnamate, | | | | | |
| commercially available as | | | | | |
| Tinogard ® TS from BASF ®) | | | | | |

TABLE 16
Deodorant Stick with Natural Wax Examples

| | Ex. J | Ex. K | Ex. L | Ex. M | Ex. N |
|---|---|---|---|---|---|
| | (wt. %) | (wt. %) | (wt. %) | (wt. %) | (wt. %) |
| Caprylic/Capric | Q.S. | Q.S. | Q.S. | Q.S. | Q.S. |
| Triglyceride | | | | | |
| Arrowroot Powder | 19 | 23 | 23 | 19 | |
| Stearyl Alcohol | 11.5 | 10.7 | 10.7 | | |
| Ozokerite | | | 1.5 | 11 | 11.75 |
| Castor Wax | 3 | 3 | 3 | | |
| Baking Soda | 12 | 12 | 6 | 6 | |
| Magnesium Hydroxide | | | 6 | 6 | 12.0 |
| Shea Butter | 2 | 2 | 2 | 2 | 2 |
| Coconut Oil | 4.5 | 4.5 | 4.5 | 4.5 | 4.5 |
| Perfume | 1-4 | 1-4 | 1-4 | 1-4 | 1-4 |
| Cyclodextrin | 4 | | | 4 | 4 |
| Tapioca Starch | | | | | 19 |

TABLE 17
Cream

| | (wt. %) |
|---|---|
| Water | Q.S. |
| Glycerin | 1-10 |
| Isohexadecane | 3.00 |
| Isopropyl Isostearate | 1.33 |
| Behenyl Alcohol | 0.25-0.75 |
| Cetyl Alcohol | 0.25-1 |
| Cetearyl Glucoside (and) Cetearyl Alcohol | 0.05-0.4 |
| (commercially available as Emulgade ® PL 68/50 from | |
| BASF ®) | |
| Stearyl Alcohol | 1-2 |
| Panthenol | 0.25-1.5 |
| Niacinamide | 0.05-2 |
| Preservative | 0.2-2 |
| Dimethicone (and) Dimethiconol (commercially | 1-2 |
| available as DC1503 from Dow ® | |
| Fragrance | 0.05-0.5 |

### Combinations:

A. A leave-on personal care composition comprising:
   a. an active ingredient;
   b. a perfume accord comprising perfume raw materials; wherein the perfume accord comprises:
      i. an odor intensity score (OIS) of ≥6, preferably ≥8, and even more ≥10, as determined by Formula I:

$$1 / n {{\sum }^{n}}_{i = 1} {C}_{i} / {M}_{i} = OIS \qquad Formula ( I )$$

wherein n is the number of perfume raw materials present in the perfume accord, Cᵢ is the concentration of the i^{th} perfume raw material in the composition, and Mᵢ is the concentration of the i^{th} perfume raw material sufficient to provide an odor intensity rating in a leave-on composition chassis of 2 on a scale from zero to five;
   ii. from about 10% to about 100%, by total weight of the perfume accord, of volatile materials each having a vapor pressure at 25 °C of less than 0.1 torr (13.3 Pa);
   iii. from about 10% to about 90%, by total weight of the perfume accord, of volatile materials each having a VP at 25 °C of less than 0.01 torr (1.33 Pa); and
   iv. from about 5% to about 70%, by total weight of the perfume accord, of volatile materials that each having a VP at 25 °C of less than 0.001 torr (0.133 Pa);
wherein the leave-on composition chassis is the leave-on composition without the perfume accord.
B. The leave-on personal care composition according to Paragraph A, wherein the perfume accord comprises from about 25% to about 98%, preferably from about 40% to about 95%, more preferably from about 50% to about 95%, and even more preferably from about 60% to about 90%, by weight of the perfume accord, of volatile materials each having a VP at 25 °C of less than 0.1 torr (13.3 Pa).
C. The leave-on personal care composition according to any one of the preceding paragraphs, wherein the perfume accord comprises ≥65%, preferably ≥70%, more preferably ≥75% more preferably ≥81%, more preferably ≥83%, and even more preferably ≥85%, by weight of the perfume, of volatile materials that each having a VP at 25 °C of less than 0.1 torr (13.3 Pa).
D. The leave-on personal care composition according to any one of the preceding paragraphs, wherein the perfume accord comprises from about 30% to about 85%, preferably from about 47% to about 80%, more preferably from about 50% to about 78%, and even more preferably from about 60% to about 75%, by weight of the perfume accord, of volatile materials each having a VP at 25 °C of less than 0.01 torr (1.33 Pa).
E. The leave-on personal care composition according to any one of the preceding paragraphs, wherein the perfume accord comprises ≥30%, preferably ≥47%, preferably ≥55%, preferably ≥60%, even more preferably ≥65%, and most preferably ≥70%, by weight of the perfume accord, of volatile materials each having a VP at 25 °C of less than 0.01 torr (1.33 Pa).
F. The leave-on personal care composition according to any one of the preceding paragraphs, wherein the perfume accord comprises from about 5% to about 60%, preferably from about 10% to about 50%, more preferably from about 15% to about 45%, and even more preferably from about 30% to about 40%, by weight of the perfume accord, of volatile materials each having a VP at 25 °C of less than 0.001 torr (0.133 Pa).
G. The leave-on personal care composition according to any one of the preceding paragraphs, wherein the perfume accord comprises ≥10%, preferably ≥20%, more preferably ≥25%, even more preferably ≥30%, and even more preferably ≥35%, by weight of the perfume accord, of volatile materials each having a VP at 25 °C of less than 0.001 torr (0.133 Pa).
H. The leave-on personal care composition according to any one of the preceding paragraphs, wherein the OIS is ≤30, preferably ≤25, more preferably ≤20, and even more preferably ≤15.
I. The leave-on personal care composition according to any one of the preceding paragraphs, wherein the composition comprises ≤4% of the perfume accord, preferably ≤3%, preferably ≤2.5%, and even more preferably ≤2%.
J. The leave-on personal care composition according to any one of the preceding paragraphs, wherein the composition comprises from about 0.05% to about 5% of the perfume accord, preferably from about 0.21% to about 4% of the perfume accord, preferably from about 0.5% to about 3.5%, preferably from about 0.75% to about 3%, more preferably from about 1% to about 2.5%, and even more preferably from about 1.1% to about 2%.
K. The leave-on personal care composition according to any one of the preceding paragraphs, wherein the perfume accord comprises from about 16.7% to about 100% esters, by weight of the perfume accord, preferably from about 20% to about 95%, more preferably from about 25% to about 90%, even more preferably from about 33% to about 85%, even more preferably from about 50% to about 80%, and most preferably from about 60% to about 75%.
L. The leave-on personal care composition according to any one of the preceding paragraphs, wherein the perfume accord can include ≥12% esters, by weight of the perfume accord, preferably ≥15%, preferably ≥16.7%, preferably ≥20%, preferably ≥30%, preferably ≥40%, more preferably ≥50%, even more preferably ≥60%, and most preferably ≥65%.
M. The leave-on personal care composition according to any one of the preceding paragraphs, wherein the perfume accord comprises at least 10 perfume raw materials, preferably at least 13, preferably at least 15, preferably at least 18, preferably at least 21, preferably at least 24, preferably at least 27, and even more preferably at least 30 perfume raw materials.
N. The leave-on personal care composition according to any one of the preceding paragraphs, wherein the leave-on personal care composition is stable according to visual detection.
O. The leave-on personal care composition according to any one of the preceding paragraphs, wherein the leave-on personal care composition has Ab of less than 0.5, preferably less than 0.25, or even more preferably less than 0.10 after storage for five days at 49 °C.
P. The leave-on personal care composition according to any one of the preceding paragraphs, wherein Mᵢ is about 1 ppm to about 1000 ppm for about 20% to about 100% of the perfume raw materials present in the perfume accord, preferably from about 30% to about 100%, preferably from about 40% to about 100%, preferably about 50% to about 100%, more preferably about 60% to about 100%, and even more preferably about 70% to about 100%.
Q. The leave-on personal care composition according to any one of the preceding paragraphs, wherein Mi is about 1 ppm to about 800 ppm, preferably about 1 ppm to about 600 ppm.
R. The leave-on personal care composition according to any one of the preceding paragraphs, wherein Cᵢ/Mᵢ is at least 0.05 for about 60% to about 100% of the perfume raw materials present in the perfume accord, more preferably from about 65% to about 100%, and even more preferably from about 75% to about 100%.
S. The leave-on personal care composition according to any one of the preceding paragraphs, wherein Ci/Mi is at least 0.2 for about 45% to about 100% of the perfume raw materials present in the perfume accord, preferably for about 50% to about 100%, more preferably from about 55% to about 100%, and even more preferably from about 60% to about 100%.
T. The leave-on personal care composition according to any one of the preceding paragraphs, wherein Ci/Mi is at least 0.3 for about 40% to about 100% of the perfume raw materials present in the perfume accord, preferably from about 45% to about 100%, more preferably from about 50% to about 100%, and even more preferably from about 55% to about 100%.
U. The leave-on personal care composition according to any one of the preceding paragraphs, wherein Ci/Mi is at least 0.4 for about 35% to about 100% of the perfume raw materials present in the perfume accord, preferably from about 45% to about 100%, and more preferably from about 52% to about 100%.
V. The leave-on personal care composition according to any one of the preceding paragraphs, wherein Ci/Mi is at least 0.5 for about 30% to about 100% of the perfume raw materials present in the perfume accord, preferably from about 40% to about 100%, and even more preferably from about 50% to about 100%.
W. The leave-on personal care composition according to any one of the preceding paragraphs, wherein Ci/Mi is at least 0.6 for about 25% to about 100% of the perfume raw materials present in the perfume accord, preferably from about 35% to about 100%, and even more preferably from about 45% to about 100%.
X. The leave-on personal care composition according to any one of the preceding paragraphs, wherein Ci/Mi is at least 0.7 for about 20% to about 100% of the perfume raw materials present in the perfume accord, preferably from about 30% to about 100%, and even more preferably from about 40% to about 100%.
Y The leave-on personal care composition according to any one of the preceding paragraphs, wherein Ci/Mi is at least 1 for about 15% to about 100% of the perfume raw materials present in the perfume accord, preferably from about 20% to about 100%, and even more preferably from about 25% to about 100%.
Z. The leave-on personal care composition according to any one of the preceding paragraphs, wherein the leave-on composition is a deodorant or antiperspirant.
AA. The leave-on personal care composition according to Paragraph Z, wherein the leave-on composition is intended for underarm and/or whole-body use.
BB. The leave-on personal care composition according to Paragraphs Z and AA, wherein the leave-on personal care composition is chosen from a solid stick, a soft solid, a roll-on, a gel, an aerosol, a cream, or mixtures thereof.
CC. The leave-on personal care composition according to Paragraphs Z-BB, wherein the composition is a solid stick or an aerosol and is substantially free or free of added water.
DD. The leave-on personal care composition according to any one of the preceding paragraphs, wherein the leave-on personal care composition is a deodorant and wherein the deodorant is substantially free of or free of aluminum.
EE. The leave-on personal care composition according to Paragraph DD, wherein the composition comprises from about 0.001% to about 20%, preferably from about 0.1% to about 10%, more preferably from about 1% to about 5% of the active chosen from piroctone olamine, lupamin, beryllium carbonate, magnesium carbonate, calcium carbonate, magnesium hydroxide, magnesium hydroxide and magnesium carbonate hydroxide, partially carbonated magnesium hydroxide, magnesium oxide, potassium carbonate, potassium bicarbonate, sodium carbonate, sodium sesquicarbonate, baking soda, hexamidine, zinc carbonate, thymol, polyvinyl formate, salycilic acid, niacinamide, or mixtures thereof.
FF. The leave-on personal care composition according to any one of the preceding paragraphs, wherein the leave on personal care composition is an antiperspirant composition comprising from about 0.1% to about 26%, preferably from about 1% to about 20%, and more preferably from about 2% to about 10% of the active; wherein the active is an aluminum salt having a formula Al₂(OH)₆₋ₐXₐ wherein X is Cl, Br, I, or NO₃, and a is about 0.3 to about 5, preferably from about 0.8 to about 2.5.
GG. The leave-on personal care composition according to any one of the preceding paragraphs, wherein the leave on composition is substantially free or free of silicone.
HH. A method of making the leave-on composition according to any one of the preceding paragraphs comprising:
   a. adding an active ingredient;
   b. adding the perfume accord;
   c. forming a leave-on composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to anything disclosed or claimed herein or that it alone, or in any combination with any other reference or references, teaches, suggests or discloses anything disclosed or claimed herein. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A leave-on personal care composition comprising:
a. an active ingredient;
b. a perfume accord comprising perfume raw materials; wherein the perfume accord comprises:
i. an odor intensity score (OIS) of ≥6, as determined by Formula I:
$$1 / n {{\sum }^{n}}_{i = 1} {C}_{i} / {M}_{i} = OIS \qquad Formula ( I )$$
wherein n is the number of perfume raw materials present in the perfume accord, Cᵢ is the concentration of the i^{th} perfume raw material in the composition, and Mᵢ is the concentration of the i^{th} perfume raw material sufficient to provide an odor intensity rating in a leave-on composition chassis of 2 on a scale from zero to five;
ii. from about 10% to about 100%, by total weight of the perfume accord, of volatile materials each having a vapor pressure at 25 °C of less than 0.1 torr;
iii. from about 10% to about 90%, by total weight of the perfume accord, of volatile materials each having a vapor pressure at 25 °C of less than 0.01 torr; and
iv. from about 5% to about 70%, by total weight of the perfume accord, of volatile materials that each having a vapor pressure at 25 °C of less than 0.001 torr;
wherein the leave-on composition chassis is the leave-on composition without the perfume accord.

2. The leave-on personal care composition of claim 1, wherein the OIS is ≥8.

3. The leave-on personal care composition of claim, wherein the OIS is ≥10.

4. The leave-on personal care composition of claim 1, wherein the perfume accord comprises from about 50% to about 95%, by weight of the perfume accord, of volatile materials each having a vapor pressure at 25 °C of less than 0.1 torr.

5. The leave-on personal care composition of claim 4, wherein the perfume accord comprises from about 50% to about 85%, by weight of the perfume accord, of volatile materials each having a vapor pressure at 25 °C of less than 0.01 torr.

6. The leave-on personal care composition of claim 5, from about 60% to about 75%, by weight of the perfume accord, of volatile materials each having a vapor pressure at 25 °C of less than 0.01 torr (1.33 Pa).

7. The leave-on personal care composition of claim 5, wherein the perfume accord comprises from about 5% to about 45%, by weight of the perfume accord, of volatile materials each having a vapor pressure at 25 °C of less than 0.001 torr (0.133 Pa).

8. The leave-on personal care composition of claim 1, wherein the leave-on personal care composition is stable according to visual detection.

9. The leave-on personal care composition of claim 1, wherein the leave-on personal care composition comprises a Ab of less than 0.5 after storage for five days at 49 °C.

10. The leave-on personal care composition of claim 9, wherein the leave-on personal care composition comprises a Ab of less than 0.25 after storage for five days at 49 °C.

11. The leave-on personal care composition of claim 1, wherein the composition comprises less than or equal to 0.001% of a fragrance allergen chosen from genzyl alcohol, 6-methyl coumarin, citral, isoeugenol, citronellol, limonene, Pinus mugo, Pinus pumila, Cedrus atlantica oil/extract, turpentine, alpha-terpinene, terpinolene, myroxylon pereirae oil/extract, rose ketones, 3-propylidenephthalide, Lippia citriodora absolute, methyl salicylate, acetyl cedrene, amyl salicylate, anethole, benzaldehyde, camphor, beta-caryophyllene, carvone, dimethyl phenethyl acetate, hexadecanolactone, hexamethylindanopyran, linalyl acetate, menthol, trimethylcyclopentenyl methylisopentenol, salicylaldehyde, santalol, sclareol, terpineol, tetramethyl acetyloctahydronaphthalenes, trimethylbenzenepropanol, vanillin, Cananga odorata oil/extract, Cinnamomum cassia leaf oil, Cinnamomum zeylanicum bark oil, Citrus aurantium flower oil, Citrus aurantium peel oil, Citrus aurantium bergamia peel oil, Citrus limon peel oil, lemongrass oil, Eucalyptus globulus oil, Eugenia caryophyllus oil, jasmine oil/extract, Juniperus virginiana oil, Laurus nobilis leaf oil, lavandula oil/extract, Mentha piperita oil, Mentha viridis leaf oil (spearmint oil), narcissus extract, Pelargonium graveolens flower oil, Pogostemon cablin oil, rose flower oil/extract, santalum album oil (sandalwood oil), eugenyl acetate, geranyl acetate, isoeugenyl acetate, and pinene, or combinations thereof.

12. The leave-on personal care composition of claim 1, wherein the composition comprises less than 4% of the perfume accord.

13. The leave-on personal care composition of claim 12, wherein the composition comprises less than or equal to 2.5% of the perfume accord.

14. The leave-on personal care composition of claim 1, wherein the perfume accord comprises greater than or equal to 16.7%, by weight of the perfume accord, esters.

15. The leave-on personal care composition of claim 1, wherein the perfume accord comprises at least 10 perfume raw materials.

16. The leave-on personal care composition of claim 15, wherein the perfume accord comprises at least 21 perfume raw materials.

17. The leave-on personal care composition of claim 1, wherein the leave-on composition is a deodorant or antiperspirant for underarm and/or whole-body use.

18. The leave-on personal care composition or claim 17, wherein the composition is a solid stick or an aerosol and is substantially free of added water.

19. The leave-on personal care composition of claim 17, wherein the composition is substantially free of aluminum.

20. A method of making a leave-on personal care composition comprising:
a. adding an active ingredient;
b. adding a perfume accord comprising perfume raw materials; wherein the perfume accord comprises:
i. an odor intensity score (OIS) of ≥6, as determined by Formula I:
$$1 / n {{\sum }^{n}}_{i = 1} {C}_{i} / {M}_{i} = OIS \qquad Formula ( I )$$
wherein n is the number of perfume raw materials present in the perfume accord, Cᵢ is the concentration of the i^{th} perfume raw material in the composition, and Mᵢ is the concentration of the i^{th} perfume raw material sufficient to provide an odor intensity rating in a leave-on composition chassis of 2 on a scale from zero to five;
ii. from about 10% to about 100%, by total weight of the perfume accord, of volatile materials each having a vapor pressure at 25 °C of less than 0.1 torr;
iii. from about 10% to about 90%, by total weight of the perfume accord, of volatile materials each having a vapor pressure at 25 °C of less than 0.01 torr (1.33 Pa); and
iv. from about 5% to about 70%, by total weight of the perfume accord, of volatile materials that each having a vapor pressure at 25 °C of less than 0.001 torr;
wherein the leave-on composition chassis is the leave-on composition without the perfume accord;
c. forming the leave-on composition.
